(19) United States
(12) Patent Application Publication
(10) Pub. No.: US 2008/0275005 A1
(43) Pub. Date: Nov. 6, 2008

(54) MITOCHONDRIALLY TARGETED ANTIOXIDANTS

(76) Inventors: Michael P. Murphy, Cambridge (GB); Robin A.J. Smith, Dunedin (NZ)

Correspondence Address: SEED INTELLECTUAL PROPERTY LAW GROUP PLLC, 701 FIFTH AVE, SUITE 5400, SEATTLE, WA, 98104, US

(21) Appl. No.: 12/109,170

(22) Filed: Apr. 24, 2008

Related U.S. Application Data
(63) Continuation-in-part of application No. 11/799,779, filed on May 2, 2007.
Application No. 11/799,779 is a continuation of application No. 11/172,916, filed on Jul. 5, 2005, now Pat. No. 7,232,809.
Application No. 11/172,916 is a continuation of application No. 10/722,542, filed on Nov. 28, 2003, now abandoned.
Application No. 10/722,542 is a continuation of application No. 10/272,914, filed on Oct. 18, 2002, now abandoned.
Application No. 10/272,914 is a continuation of application No. 09/968,838, filed on Oct. 3, 2001, now abandoned.
Application No. 09/968,838 is a continuation of application No. 09/577,877, filed on May 25, 2000, now Pat. No. 6,331,532.
Application No. 09/577,877 is a continuation-in-part of application No. PCT/NZ98/00173, filed on Nov. 25, 1998.
Application No. PCT/NZ98/00173 is a continuation-in-part of application No. 10/568,655, filed on Aug. 31, 2006.
Application No. 10/568,655 is a continuation-in-part of application No. 10/568,654, filed on Feb. 22, 2007.

(30) Foreign Application Priority Data
Aug. 22, 2003 (NZ) 527800
Oct. 23, 2003 (NZ) 529153
Jun. 14, 2004 (NZ) 533555
Jun. 14, 2004 (NZ) 533556

(51) Int. Cl. A61K 31/665; A61P 35/00; A61P 3/10

(52) U.S. Cl. 514/100

(57) ABSTRACT

The invention provides mitochondrially targeted antioxidant compounds. A compound of the invention comprises a lipophilic cation covalently coupled to an antioxidant moiety. In preferred embodiments, the lipophilic cation is the triphenyl phosphonium cation, and the compound is of the formula P(Ph₃)+XR.Z- where X is a linking group, Z is an anion and R is an antioxidant moiety. Also provided are pharmaceutical compositions containing the mitochondrially targeted antioxidant compounds, and methods of therapy or prophylaxis of patients who would benefit from reduced oxidative stress, which comprise the step of administering the compounds of the invention.

### CROSS-REFERENCE TO RELATED APPLICATION

This application is a continuation-in-part of U.S. patent application Ser. No. 11/799,779, filed May 2, 2007, now pending, which is a Continuation of U.S. Ser. No. 11/172,916, filed Jul. 5, 2005, which issued as U.S. Pat. No. 7,232,809 on Jun. 19, 2007, and which is a Continuation of U.S. Ser. No. 10/722,542, filed Nov. 18, 2003, now abandoned; which is a Continuation of U.S. Ser. No. 10/272,914, filed Oct. 18, 2002, now abandoned; which is a Continuation of U.S. Ser. No. 09/968,838, filed Oct. 3, 2001, now abandoned; which is a Continuation of U.S. Ser. No. 09/577,877, filed May 25, 2000, which issued as U.S. Pat. No. 6,331,532 on Dec. 18, 2001, and which is a Continuation-in-Part of PCT application PCT/NZ98/00173, filed Nov. 25, 1998, all of which applications are incorporated herein by reference in their entirety.

This application is also a continuation-in-part of U.S. Ser. No. 10/568,655 which is a filing made under 35 U.S.C. §371 based on PCT/NZ2004/000196, filed Aug. 24, 2004, which claims priority to New Zealand Application Nos. 533556, filed Jun. 14, 2004; 529153, filed Oct. 23, 2003; and 527800, filed Aug. 22, 2003, all of which applications are incorporated herein by reference in their entirety.

This application is also a continuation-in-part of U.S. Ser. No. 10/568,654 which is a filing made under 35 U.S.C. §371 based on PCT/NZ2004/000197, filed Aug. 23, 2004, which claims priority to New Zealand Application Nos. 533555, filed Jun. 14, 2004; 529153, filed Oct. 23, 2003; and 527800, filed Aug. 22, 2003, all of which applications are incorporated herein by reference in their entirety.

### BACKGROUND OF THE INVENTION

1. Field of the Invention

The invention relates to antioxidants having a lipophilic cationic group and to uses of these antioxidants, for example, as pharmaceuticals.

2. Description of the Related Art

Oxidative stress contributes to a number of human degenerative diseases associated with aging, such as Parkinson's disease, and Alzheimer's disease, as well as to Huntington's Chorea, diabetes and Friedreich's Ataxia, and to non-specific damage that accumulates with aging (e.g., Shigenaga et al., 1994 Proc Nat. Acad. Sci. USA 91:10771; Miquel et al., 1980 Exp Gerontol. 15:575). It also contributes to inflammation and ischemic-reperfusion tissue injury in stroke and heart attack (e.g., Zweier et al., 1988 J. Biol. Chem. 263:1353; Zweier et al., 1987 Proc. Nat. Acad. Sci. USA 84:1404), and also during organ transplantation and surgery. To prevent the damage caused by oxidative stress in these and other diseases, a number of antioxidant therapies have been developed. However, most of these therapies are not targeted within cells and are therefore less than optimally effective.

Mitochondria are intracellular organelles responsible for energy metabolism. Consequently, mitochondrial defects are damaging, particularly to neural and muscle tissues which have high energy demands. They are also the major source of the free radicals and reactive oxygen species that cause oxidative stress inside most cells. At the time of filing the priority applications on which the present continuation-in-part application is based, elevated (i.e., increased in a statistically significant manner relative to appropriate controls) oxidative stress, including elevated mitochondrial oxidative stress, was known as an etiologic factor in a large number of diseases and clinical conditions (e.g., Halliwell and Gutteridge, Free Radicals in Biology and Medicine (3^{rd} Ed.), Oxford University Press, Oxford, UK, 1999, pages 617-783).

For example, a number of liver diseases are characterized by elevated oxidative stress, which in many cases is elevated mitochondrial oxidative stress. Liver diseases characterized by elevated oxidative stress include fatty liver disease, hepatic viral infection, alcoholic liver disease, transplant-associated liver inflammation and liver cancer (e.g., hepatocellular carcinoma, HCC). Such liver diseases are frequently accompanied by liver inflammation, which can lead to liver fibrosis, cirrhosis and, finally, end-stage liver failure.

Oxidative stress has been reported as a factor in fatty liver disease (e.g., Leclerq et al (April 2000) J Clin Invest. 105:1067-1075; Lavine et al. (2000) J. Pediatr. 136:734-8; Pessayre et al. (1999) Cell Biol Toxicol. 15(6):367-73; Caldwell et al. (1999) J. Hepatol. 31:430-34; Bonkovsky et al (September 1999) J. Hepatol. 31(3):421-9; Diehl (1999) Semin Liver Dis. 19(2):221-9; Day et al. (1998) Gastroenterol. 114:842-5; Berson et al (1998) Gastroenterol. 114(4):764-74; Letteron et al (1996) J. Hepatol. 24(2):200-8; Fromenty et al (1995) Pharmacol. Ther. 67:101-154). Non-alcoholic fatty liver disease, or NAFLD, impacts approximately 75-110 million people in the United States. The disease is highly correlated to obesity, with 25% of the U.S. population having a body mass index (BMI)>30. Accumulation of fat in the liver results in inflammation, which can be detected by the use of liver enzyme tests, principally alanine aminotransferase (ALT). As the disease progresses, the inflammation leads to scarring and fibrosis of the liver, resulting in non-alcoholic steatohepatitis, or NASH. Approximately 3-5% of the U.S. population, or 9-15 million people, have NASH. Of these NASH patients, approximately 30% will progress to cirrhosis, 5% will develop liver cancer, and 2.5% will go on to have a liver transplant. Additionally, in the U.S., approximately two million people have alcoholic liver disease, which in some cases is accompanied by fat accumulation in the liver, and for which oxidative stress has also been identified as a factor in the pathogenesis of disease (e.g., Crabb, 1999 Keio J. Med. 48:184).

Oxidative stress has also been described as a factor in viral infections of the liver such as by hepatitis C virus (HCV) (e.g., Barbaro et al (1999) Am. J. Gastroenterol. 94. 2198-2205; Larrea et al. (1998) Free Radic Biol Med. (7-8):1235-41. Yamamoto et al. (June 1998) Biochem Biophys Res Commun. 247(1):166-70; Bonkovsky (September 1997) Hepatology 26(3 Suppl 1):143S-151 S; Von Herbay et al (December 1997) Free Radic Res. 27(6):599-605. Houglum et al (1997) Gastroenterology 113. 1069-1073. Schwarz (1996) Free Radic Biol Med. 21(5):641-9, and in alcoholic liver disease (e.g., Bailey et al., 1998 Hepatol. 28:1318, Bailey et al. 1999 Alc. Clin. Exp. Res. 23:1210).

Among such viral infections of the liver, viral hepatitis represents a significant cause of liver inflammation characterized by oxidative stress. A variety of viruses attack the liver, but the most prevalent and medically daunting infection comes from hepatitis C virus (HCV). There are approximately 170 million people infected with HCV worldwide. It is estimated that approximately 7.4 million people in the U.S. and the five largest European countries are infected with the hepatitis C virus. However, it is estimated that only about 2-3% of these infected people are currently being treated, due to under-diagnosis and poor availability of treatment options: only about 50% of treated patients respond to ribavirin and interferon combination therapy. The number of patients in need of treatment is likely to grow rapidly in coming years, due in part to the increasing use of improved rapid diagnostics and the need for an effective therapy targeted at patients not responding to ribavirin/interferon. Without effective treatment, these patients, much like the NAFLD and NASH patients, will progress to liver fibrosis, cirrhosis, and cancer.

In end-stage liver disease, the last treatment option is liver transplantation. The difficulty with this approach is finding a suitably tissue-matched organ for the patient. It is estimated currently that approximately 17,000 people are awaiting liver transplantation, while only 5,300 transplants were performed in the U.S. in 2002. Liver inflammation that results from oxidative stress can also be present in the course of liver transplantation (e.g., Nakano et al., 1996 Eur Surg. Res. 28:245; Biasi et al., 1995 Free Rad. Biol. Med. 19:311; Galley et al., 1995 Clin. Sci. (Lond) 89:329; Goode et al., 1994 Hepatol. 19:354). In other transplantation contexts, such as bone marrow transplant or blood platelet transfusion, deleterious oxidative stress contributes to cell and/or tissue damage (e.g., Durken et al., 1995 Bone Marrow Transplant. 15:757; Pich et al., 2002 Free Radical Res 36(4):429).

In the context of cancer, too, oxidative stress is also a significant contributing factor that has been identified in mechanisms of carcinogenesis, mitochondrial damage, aberrant apoptosis, and malignancy (e.g., Kroemer et al (1998) Annu. Rev. Physiol. 60:619-642; Modica-Napolitano et al (2001) Adv Drug Delivery Rev 49:63-70; Murphy (1997) Trends Biotechnol. 15(8):326:30; Dreher et al (1996) Eur J Cancer 32A(1):30-8; Slaga (1995) Adv Exp Med. Biol. 369:167-74; Toyokuni et al (1995) FEBS Lett. 358(1):1-3; Clemens (1991) Klin Wochenschr. 69(21-23):1123-34; Goldstein et al (1990) Free Radic Res Commun. 11 (1-3):3-10; Perchellet et al (1989) Free Radic Biol Med. 7(4):377-408). Also with regard to cancer, it has been recognized for some time that the anthracycline class of antineoplastic agents can trigger oxidative stress that manifests in the form of free radical-induced damage to cardiac myocytes, causing anthracyline-induced cardiotoxicity (e.g., Shan et al., 1996 Ann. Int. Med. 125:47).

Elevated oxidative stress, including elevated mitochondrial oxidative stress, is also an etiologic factor in cardiometabolic syndrome, a convergence of multiple risk factors that puts a person at significantly higher risk for morbidity and mortality from cardiovascular disease (CVD). (e.g., Schmidt et al., 1996 Diabetes Care 19:414; Hulthe et al., 2000 Arteroscler Thromb Vasc Biol 20:2140; Rantala et al., 1999 J Intern Med. 245:163; Bonora et al., 1998 Diabetes 47:1643; Liese et al., 1997 Ann Epidemiol. 7:407; Haffner et al., 1997 Diabetes 46:63; Meigs et al., 1997 Diabetes 46:1594; Kannel, 2000 Am J. Hypertens. 13(1 pt 2):3S; See also NIH, Third Report of the National Cholesterol Education Program Expert Panel on Detection, Evaluation, and Treatment of High Blood Cholesterol in Adults (Adult Treatment Panel III). Bethesda, Md.; National Institutes of Health; Publication 01-3670; 2001). Focused around obesity, in combination with elevated blood lipids, blood pressure, and/or insulin resistance, impacted people are 3-4 times more likely to die from CVD than people without these combined risk factors. In the United States, it is estimated that approximately 47 million people suffer from cardiometabolic syndrome. Untreated, patients can go on to develop Type II diabetes (18 million in the US), stroke (6 million in US), and coronary artery disease (13 million in the US).

Other recognized manifestations of CVD characterized by elevated oxidative stress include cardiovascular hypertension, atherosclerosis and heart failure (e.g., Zalba et al (2000) J Physiol Biochem. 56(1):57-64; Touyz (1999) Curr Hypertens Rep. 2(1):98-105; Frei (1999) Proc Soc Exp Biol Med. 222(3):196-204; Romero et al (1999) Hypertension 34(4 Pt 2):943-9; Harrison (1997) Clin Cardiol. 20(11 Suppl 2):11-11-7; Vogel (1997) Clin Cardiol. 20(5):429-32; Romero-Alvira et al (1996) Med. Hypotheses. 46(4):414-20).

Various lung diseases are associated with oxidative stress, including obstructive pulmonary disease, chronic obstructive pulmonary disease (COPD), cystic fibrosis, emphysema, pulmonary fibrosis, adult respiratory distress syndrome, pulmonary hypertension and asbestosis, and further including these and other lung diseases (e.g., lung cancer) that may be caused by smoking. See, e.g., Camhi et al., New Horiz. 1995 May; 3(2):170-82; Quinlan et al. Environ Health Perspect. 1994 June; 102 Suppl 2:79-87; Datta et al. Natl. Med. J. India 2000 November December; 13(6):304-10; Doelman et al., Free Radic Biol Med. 1990; 9(5):381-400; Panus et al., Exp Lung Res. 1988 14 Suppl:959-76; Wright et al., Environ Health Perspect. 1994 December; 102 Suppl 10:85-90; Poli et al. Free Radic Biol Med. 1997; 22(1-2):287-305; Chow, Ann N Y Acad Sci. 1993 May 28; 686:289-98; Rahman et al., Free Radic Biol Med. 1996; 21(5):669-81; Kamp et al. Free Radic Biol Med. 1992; 12(4):293-315; Gonzalez et al., 1996 Shock 6 Suppl 1:S23-6; Konstan M W, Berger M. Infection and inflammation in the lung in cystic fibrosis In: Davis P B, ed. Cystic Fibrosis New York, Marcel Dekker, 1993; pp. 219-276; Brown et al. Thorax 1994; 49:738-742; Doring G, Knight R, Bellon G. Immunology of cystic fibrosis In: Hodson M, Geddes D, editors. Cystic Fibrosis London, Chapman & Hall, 1994; pp. 99-129; Herget et al. Physiol Res. 2000; 49(5):493-501; MacNee Chest 2000 May; 117(5 Suppl 1):303S-17S; Van Klayeren et al. Curr Opin Pulm Med. 1999 March; 5(2):118-23.

Diabetes mellitus is a metabolic disorder in humans with a prevalence of approximately one percent in the general population, with one-fourth of these being the Type 1, insulin-dependent “early onset” (usually before age 30 years in humans) category (Foster, D. W., Harrison's Principles of Internal Medicine, Chap. 114, pp. 661-678, 10th Ed., McGraw-Hill, New York). The disease manifests itself as a series of hormone-induced metabolic abnormalities which eventually lead to serious, long-term and debilitating complications involving several organ systems including the eyes, kidneys, nerves, and blood vessels. Type 2 diabetes mellitus, or “late onset” diabetes, is a common, degenerative disease affecting 5 to 10 percent of the population in developed countries. The propensity for developing type 2 diabetes mellitus (“type 2 DM”) is reportedly maternally inherited, suggesting a mitochondrial genetic involvement. (Alcolado et al., Br. Med. J. 302:1178-1180 (1991); Reny, S. L., International J. Epidem. 23:886-890 (1994)).

Oxidative stress is associated with diabetes mellitus, including Type 1 DM and Type 2 DM. (e.g., West (2000) Diabet Med. 17(3):171-80; Dominguez et al (1998) Diabetes Care 21(10):1736-42; Rosen et al (1998) Mol Cell Biochem. 188(1-2):103-11; De Mattia et al (1998) Diabetologia 41(11):1392-6; Low et al (1997) Diabetes 46 Suppl 2:S38-42; Kubisch et al (1997) Diabetes 46(10):1563-6; Kubisch et al (1994) PNAS Vol. 91(21):9956-9) Elevated reactive oxygen species (ROS) have been implicated in the pathogenesis of Type 1 diabetes (e.g., Hannon-Fletcher et al., 2000 Mutat. Res. 460:53; Ho et al., 1999 Proc Soc Exp Biol. Med. 222:205) and Type 2 diabetes (e.g., Rosen et al., 2001 Diabetes Metab Res Rev. 17:189, including references cited therein; see also references cited above). At the cellular level, the degenerative phenotype characteristic of late onset (Type 2) diabetes mellitus includes altered mitochondrial respiratory function, impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, circulating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies (e.g., Jakus, 2000 Bratisl. Lek Listy 101:541; Giugliano et al., 1995 Metabolism 44:363), peripheral and sensory neuropathies and blindness.

Oxidative stress, including oxidative damage deriving from mitochondrial dysfunction and reactive free radical generation, contributes to the pathogenesis of a wide variety of neurodegenerative diseases, including Alzheimer's Disease, amyotrophic lateral sclerosis, Huntington's Disease, Parkinson's Disease, Freidreich's Ataxia, multiple sclerosis and others. See, e.g., Sun et al (1998) J Biomed Sci. 5(6):401-14; Beal (1998) Biochim Biophys Acta. August 10; 1366(1-2):211-23; Simon ian et al (1996) Annu Rev Pharmacol Toxicol. 36:83-106; Gorman et al (1996) J Neurol Sci. 139 Suppl:45-52; Beal (1996) Curr Opin Neurobiol. 6(5):661-6; Ince et al (1998) Neuropathol Appl Neurobiol. 24(2):104-17; Louvel et al (1997) Trends Pharmacol Sci. 18(6):196-203; Oteiza et al (1997) Neurochem Res. 22(4):535-9; Gurney et al (1996) Ann Neurol. 39(2):145-6; Bergeron (1995) J Neurol Sci. 129 Suppl:81-4; Beal (1995) Ann Neurol 38(3):357-66; Tritschler et al (1994) Biochem Mol Biol Int. 34(1):169-81; Olanow et al (1994) Curr Opin Neurol. 7(6):548-58; Browne et al (1999) Brain Pathol 9(1):147-63; Polidori et al (1999) Neurosci Lett. 272(1):53-6; Shapira (1997) Ann Neurol. 41(2):141-2; Browne et al (1997) Ann Neurol. 41(5):646-53; Feigin et al (1996) Mov Disord 11 (3):321-3; Borlongon et al (1996) J Fla Med Assoc. 83(5):335-41; Nakao et al (1996) Neuroscience 73(1):185-200; Peyser et al (1995) Am J Psychiatry 152(12):1771-5; Beal, Howell and Bodis-Wollner (Eds.), Mitochondria & Free Radicals in Neurodegenerative Diseases, 1997, Wiley-Liss, NY.

Oxidative stress has also been found to be a component of sepsis, a systemic inflammatory reaction (septicemia) that may be triggered by infections, trauma or pancreatitis, leading to multiple organ dysfunction and/or hypotension. Mitochondrial oxidative damage to the heart and liver are major components of the pathology due to excessive inflammation during sepsis. Bacterial endotoxin can trigger sepsis or septic shock, also referred to as endotoxic shock, with inflammatory mechanisms inducing free radical oxidative injury leading to organ failure. (e.g., Das et al., 2000 Crit. Care 4:290; Base et al, 1998 FEBS Lett. 438:159; Oldham et al., 1998 J Am Diet Assoc 98:1001; Kantrow et al., 1997 Arch Bioch Biophys 345:278; Galley et al. 1997 Free Rad Biol Med 23:768; Taylor et al., 1995 J Crit. Care 10:122; Taylor et al., 1995 Arch Bioch Biophys 316:70.)

Certain opthalmological disorders, and in particular, macular degeneration, retinal degeneration and other diseases in which retinal cells such as retinal pigmented epithelial (RPE) cells, involve oxidative damage resulting from aberrant oxidative stress. See, e.g., Nicolas et al (1996) Exp Eye Res. 62(4):399-408; Christen et al (January 1996) Ann Epidemiol. 6(1):60-66; Snodderly Am J Clin Nutr. 62(6 Suppl):1448S-1461S; Kutty et al (1995) PNAS 92(4):1177-81; Christen (September 1994) Am J. Med. 97(3A):14S-17S.

Accordingly it has been appreciated that patients having, for example, a human degenerative disease associated with aging, non-specific cell, tissue or organ damage that accumulates with aging, inflammation, ischemia-reperfusion tissue injury accompanying at least one of stroke, heart attack, organ transplantation and surgery, diabetes, neurodegenerative disease, or cancer, would benefit from reduced oxidative stress, as would patients having other diseases associated with oxidative stress, if effective, appropriately targeted antioxidant compositions were available.

Lipophilic cations may be accumulated in the mitochondrial matrix because of their positive charge (Rottenberg, (1979) Methods Enzymol, 55, 547-560; Chen, (1988) Annu Rev Cell Biol 4, 155-181). Such ions are accumulated provided they are sufficiently lipophilic to screen the positive charge or delocalize it over a large surface area, also provided that there is no active efflux pathway and the cation is not metabolized or immediately toxic to a cell.

The present application makes explicit what was implicit in the related priority filings, with respect to what the skilled person would at the time of such filings have recognized are some of the particular diseases with which oxidative stress, including mitochondrial oxidative stress, is associated.

### BRIEF SUMMARY OF THE INVENTION

In its broadest aspect, the invention provides a mitochondrially-targeted antioxidant which comprises a lipophilic cation covalently coupled to an antioxidant moiety, wherein the antioxidant moiety is capable of being transported through the mitochondrial membrane and accumulated within the mitochondria of intact cells, with the proviso that the compound is not thiobutyltriphenylphosphonium bromide.

Preferably, the lipophilic cation is the triphenylphosphonium cation.

Preferably, the mitochondrially-targeted antioxidant has the formula

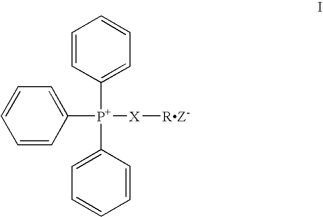

wherein Z is an anion, X is a linking group and R is an antioxidant moiety.

Preferably, X is a C₁-C₃₀, more preferably C₁-C₂₀, carbon chain, optionally including one or more double or triple bonds, and optionally including one or more substituents (such as hydroxyl, carboxylic acid or amide groups) and/or unsubstituted or substituted alky, alkenyl or alkynyl side chains.

Preferably, X is (CH₂)ₙ, where n is an integer of from 1 to 20, more preferably of from about 1 to 15.

More preferably, X is an ethylene, propylene, butylene, pentylene or decylene group.

Preferably, Z is a pharmaceutically acceptable anion, a number of which are known to those familiar with the art, and which in certain preferred embodiments may be an alkyl sulfonate, for example, methanesulfonate, p-toluenesulfonate, ethanesulfonate, benzenesulfonate, 2-naphthalenesulfonate or other alkyl sulfonate, or another pharmaceutically acceptable anion. These and other pharmaceutically acceptable anions are described in PCT/NZ2004/000196 and PCT/NZ2004/000197.

In one particularly preferred embodiment, the mitochondrially-targeted anti-oxidant of the invention has the formula

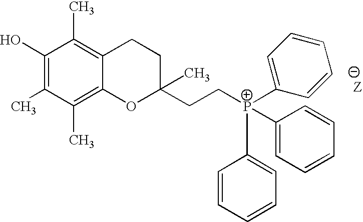

including all stereoisomers thereof.

In certain embodiments, Z is a pharmaceutically acceptable anion such as Br and in more preferred embodiments Z is a pharmaceutically acceptable anion that is not a bromide ion or a nitrate anion and does not exhibit reactivity against the antioxidant moiety, as described in PCT/NZ2004/000196 and PCT/NZ2004/000197, for example, an alkyl sulfonate such as methanesulfonate, p-toluenesulfonate, ethanesulfonate, benzenesulfonate, 2-naphthalenesulfonate or other alkyl sulfonate. The above compound is referred to herein as “compound 1”.

In another preferred embodiment, the mitochondrially-targeted antioxidant has the general formula:

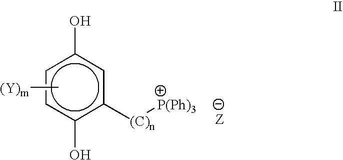

wherein:

Z is a pharmaceutically acceptable anion as discussed herein and in PCT/NZ2004/000196 and PCT/NZ2004/000197, which in certain embodiments may be a halogen, while in certain other more preferred embodiments Z is a pharmaceutically acceptable anion that is not a bromide ion or a nitrate anion and does not exhibit reactivity against the antioxidant moiety, for example, an alkyl sulfonate such as methanesulfonate, p-toluenesulfonate, ethanesulfonate, benzenesulfonate, 2-naphthalenesulfonate or other alkyl sulfonate,

m is an integer from 0 to 3,

each Y is independently selected from groups, chains and aliphatic and aromatic rings having electron donating and accepting properties,

(C)ₙ, represents a carbon chain optionally including one or more double or triple bonds, and optionally including one or more substituents and/or unsubstituted or substituted alkyl, alkenyl or alkynyl side chains, and

n is an integer of from 1 to 20 such as 2, 3, 4, 5, 6, 7, 8, 9, 10, 11, 12, 13, 14, 15, 16, 17, 18, or 19.

Preferably, each Y is independently selected from the group consisting of alkoxy, thioalkyl, alkyl, haloalkyl, halo, amino, nitro, optionally substituted aryl, or, when m is 2 or 3, two Y groups, together with the carbon atoms to which they are attached, form an aliphatic or aromatic carbocyclic or heterocyclic ring fused to the aryl ring. More preferably, each Y is independently selected from methoxy and methyl.

Preferably, (C)ₙ, is an alkyl chain of the formula (CH₂)ₙ.

In a particularly preferred embodiment, the mitochondrially-targeted antioxidant of the invention has the formula

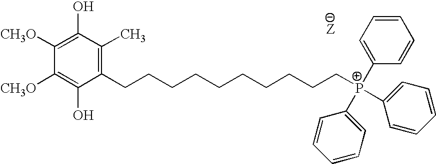

In certain embodiments Z is Br, while in certain other more preferred embodiments Z is a pharmaceutically acceptable anion that is not a bromide ion or a nitrate anion and does not exhibit reactivity against the antioxidant moiety as discussed herein and in PCT/NZ2004/000196 and PCT/NZ2004/000197, for example, an alkyl sulfonate such as methanesulfonate, p-toluenesulfonate, ethanesulfonate, benzenesulfonate, 2-naphthalenesulfonate or other alkyl sulfonate. The above compound is referred to herein as “mitoquinol”. The oxidized form of the compound is referred to as “mitoquinone”.

In a further embodiment, the present invention provides a pharmaceutical composition suitable for treatment of a patient who would benefit from reduced oxidative stress which comprises an effective amount of a mitochondrially-targeted antioxidant of the present invention in combination with one or more pharmaceutically acceptable carriers or diluents.

In another embodiment, the invention provides a method of reducing oxidative stress in a cell which comprises the step of administering to said cell a mitochondrially targeted antioxidant as described herein.

In another embodiment, the invention provides a method of therapy or prophylaxis of a patient who would benefit from reduced oxidative stress which comprises the step of administering to said patient a mitochondrially-targeted antioxidant as described herein.

Accordingly in certain embodiments there is provided a method of therapy or prophylaxis of a patient who would benefit from reduced oxidative stress, comprising administering to the patient a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the patient has a disease that is selected from (i) a human degenerative disease associated with aging, (ii) non-specific cell, tissue or organ damage that accumulates with aging, (iii) inflammation, (iv) ischemia-reperfusion tissue injury accompanying at least one of stroke, heart attack, organ transplantation and surgery, (v) diabetes, (vi) neurodegenerative disease, and (vii) cancer.

In another embodiment there is provided a method of therapy or prophylaxis of a patient who would benefit from reduced oxidative stress, comprising administering to the patient a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the patient has a disease that is selected from (i) a liver disease characterized by elevated oxidative stress, (ii) a cardiometabolic syndrome condition characterized by elevated oxidative stress, (iii) a cardiovascular disease characterized by elevated oxidative stress, (iv) macular or retinal degeneration, (v) anthracycline-induced cardiotoxicity, (vi) sepsis, and (vii) a lung disease characterized by elevated oxidative stress.

In another embodiment there is provided a method of treating or preventing a disease associated with oxidative stress, comprising administering a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the disease associated with oxidative stress is selected from (i) a human degenerative disease associated with aging, (ii) non-specific cell, tissue or organ damage that accumulates with aging, (iii) inflammation, (iv) ischemia-reperfusion tissue injury accompanying at least one of stroke, heart attack, organ transplantation and surgery, (v) diabetes, (vi) neurodegenerative disease, and (vii) cancer.

In another embodiment there is provided a method of treating or preventing a disease associated with oxidative stress, comprising administering a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the disease associated with oxidative stress is selected from (i) a liver disease characterized by elevated oxidative stress, (ii) a cardiometabolic syndrome condition characterized by elevated oxidative stress, (iii) a cardiovascular disease characterized by elevated oxidative stress, (iv) macular or retinal degeneration, (v) anthracycline-induced cardiotoxicity, (vi) sepsis, and (vii) a lung disease characterized by elevated oxidative stress.

In certain further embodiments oxidative stress comprises mitochondrial oxidative stress. In certain other further embodiments (i) the human degenerative disease associated with aging is selected from Parkinson's disease and Alzheimer's disease, (ii) the inflammation is caused by sepsis or septic shock, (iii) the diabetes comprises at least one condition that is selected from type 1 diabetes, type 2 diabetes, impaired glucose tolerance and a diabetic complication, (iv) the neurodegenerative disease is selected from amyotrophic lateral sclerosis, Parkinson's disease, Huntington's disease, Alzheimer's disease, Freidreich's ataxia and traumatic brain injury, and (v) the cancer comprises hepatocellular carcinoma. In certain further embodiments sepsis or septic shock comprises endotoxic shock. In certain other further embodiments the diabetic complication comprises diabetic neuropathy. In certain other further embodiments the liver disease characterized by elevated oxidative stress is selected from a fatty liver disease, a hepatic viral infection, alcoholic liver disease, transplantation-associated liver inflammation and liver cancer. In certain further embodiments the fatty liver disease is selected from non-alcohol induced steatohepatitis, non-alcohol-induced fatty liver disease, and alcohol-induced steatohepatitis. In certain still further embodiments the hepatic viral infection comprises a hepatitis C virus (HCV) infection. In certain other still further embodiments the liver cancer comprises hepatocellular carcinoma.

In certain embodiments the cardiovascular disease characterized by elevated oxidative stress comprises one or more of cardiovascular hypertension, atherosclerosis and heart failure. In certain other embodiments the lung disease characterized by elevated oxidative stress is selected from obstructive pulmonary disease, cystic fibrosis, emphysema, pulmonary fibrosis, adult respiratory distress syndrome, pulmonary hypertension and asbestosis. In certain further embodiments the obstructive pulmonary disease is chronic obstructive pulmonary disease.

These and other aspects of the invention will be evident upon reference to the present disclosure including the following detailed description and the attached drawings. All of the U.S. patents, U.S. patent application publications, U.S. patent applications, foreign patents, foreign patent applications and non-patent publications referred to in this specification and/or listed in the Application Data Sheet, are incorporated herein by reference in their entirety, as if each was incorporated individually. Aspects of the invention can be modified, if necessary, to employ concepts of the various patents, applications and publications to provide yet further embodiments of the invention.

### BRIEF DESCRIPTION OF THE SEVERAL VIEWS OF THE DRAWING(S)

In particular, a better understanding of the invention will be gained with reference to the accompanying drawings, in which:

FIG. 1 is a graph which shows the uptake by isolated mitochondria of compound 1, a mitochondrially-targeted antioxidant according to the present invention;

FIG. 2 is a graph which shows the accumulation of compound 1 by isolated mitochondria;

FIG. 3 is a graph which shows a comparison of a compound 1 uptake with that of the triphenylphosphonium cation (TPMP);

FIG. 4 is a graph which shows that compound 1 protects mitochondria against oxidative damage;

FIG. 5 is a graph which compares compound 1 with vitamin E and the effect of uncoupler and other lipophilic cations;

FIG. 6 is a graph which shows that compound 1 protects mitochondrial function from oxidative damage;

FIG. 7 is a graph which shows the effect of compound 1 on mitochondrial function;

FIG. 8 is a graph which shows the uptake of compound 1 by cells;

FIG. 9 is a graph which shows the energisation-sensitive uptake of compound 1 by cells; and

FIG. 10 is a graph which shows the effect of compound 1 on cell viability.

FIG. 11 shows the UV-absorption spectrum of [10-(6′-ubiquinonyl)decyltriphenylphosphonium bromide] (herein referred to as “mitoquinone”) and of the reduced form of the compound [10-(6′-ubiquinonyl)decyltriphenylphosphonium bromide] (herein referred to as “mitoquinol”).

FIGS. 12A to 12D show reactions of [10-(6′-ubiquinonyl)decyltriphenylphosphonium bromide] (“mitoquinone”) and the reduced form of the compound (“mitoquinol”) with mitochondrial membranes. Beef heart mitochondrial membranes (20 μg/ml) were suspended in 50 mM sodium phosphate, pH 7.2 at 20° C. In panel A rotenone and antimycin were present and for the t=0 scan, then succinate (5 mM) was added and scans repeated at 5 minute intervals as indicated. In panel B A₂₇₅ was monitored in the presence of rotenone and antimycin and then mitoquinone (50 μM) was added, followed by succinate (5 mM) and malonate (20 mM) where indicated. In Panel C rotenone, ferricytochrome c (50 μM) and malonate (20 mM) were present, A₂₇₅ was monitored and mitoquinol (50 μM) and myxathiazol (10 μM) were added where indicated. In panel D A₅₅₀ was monitored and the experiment in Panel C was repeated in the presence of KCN. Addition of myxathiazol inhibited this rate by about 60-70%. There was no reaction between mitoquinone and succinate or NADH in the absence of mitochondrial membranes, however mixing 50 μM mitoquinone, but not mitoquinol, with 50 μM ferricytochrome c led to some reduction of A₅₅₀;

FIG. 13 shows reactions of mitoquinol and mitoquinone with pentane-extracted mitochondrial membranes. Pentane extracted beef heart mitochondria (100 μg protein/ml) were suspended in 50 mM sodium phosphate, pH 7.2 at 20° C. In Panel A NADH (125 μM) was added and A₃₄₀ was monitored and ubiquinone-1 (UQ-1; 50 μM) added where indicated. This was repeated in Panel b, except that mitoubiquinone (50 μM) was added. In Panel C pentane extracted mitochondria were incubated with mitoquinone (50 μM), A₂₇₅ was monitored and succinate (5 mM) and malonate (20 mM) added where indicated. In Panel D pentane-extracted mitochondria were incubated with NADH (125 μM), ferricytochrome c (50 μM) and A₅₅₀ was monitored and mitoquinone (50 μM) was added where indicated. Addition of myxathiazol inhibited the rate of reduction by about 60-70%;

FIG. 14 shows reduction of mitoquinone by intact mitochondria. Rat liver mitochondria (100 μg/ml) were incubated in 120 mM KCl, 10 mM HEPES, 1 mM EGTA, pH 7.2 at 20° C. and A₂₇₅ monitored. In panel A rotenone and succinate (5 mM) were present and mitoquinone (50 μM) was added where indicated. This experiment was repeated in the presence of malonate (20 mM) or FCCP (333 nM). In panel B glutamate and malate (5 mM of each) were present from the start and mitoquinone (50 μM) was added where indicated. This experiment was repeated in the presence of FCCP or with rotenone and FCCP. Addition of TPMP (50 μM) instead of mitoquinone did not lead to changes in A₂₇₅;

FIG. 15 shows uptake of radiolabelled mitoquinol by energized rat liver mitochondria and its release on addition of the uncoupler FCCP; and

FIG. 16 shows the effect of mitoquinol on isolated rat liver mitochondria. In A rat liver mitochondria energized with succinate were incubated with various concentrations of mitoquinol and the membrane potential determined as a percentage of control incubations. In B the respiration rate of succinate energized mitochondria under state 4 (black), state 3 (white) and uncoupled (stippled) conditions, as a percentage of control incubations.

FIG. 17 shows plasma alanine aminotransferase (ALT) levels in chronically HCV-infected subjects receiving indicated dose of 10-(6′-ubiquinonyl) decyltriphenylphosphonium methanesulfonate or placebo during (“treatment”) and after (“follow-up”) a treatment regimen. FIG. 17A shows mean plasma ALT over time. FIG. 17B shows the percentage change in plasma ALT level for each treatment group at day 28, compared to baseline. FIG. 17C shows the absolute change in plasma ALT level for each treatment group at day 28, compared to baseline.

FIG. 18 shows HCV viral load as determined from HCV RNA detection in chronically HCV-infected subjects receiving indicated dose of 10-(6′-ubiquinonyl) decyltriphenylphosphonium methanesulfonate or placebo during a treatment regimen.

FIG. 19 shows mean change in plasma aspartate aminotransferase (AST) levels in chronically HCV-infected subjects receiving indicated dose of 10-(6′-ubiquinonyl) decyltriphenylphosphonium methanesulfonate or placebo during a treatment regimen.

FIG. 20 shows effects of 10-(6′-ubiquinonyl) decyltriphenyl-phosphonium methanesulfonate on systolic blood pressure in a stroke-prone rat model of hypertension.

FIG. 21 shows effects of 10-(6′-ubiquinonyl) decyltriphenyl-phosphonium methanesulfonate on bioavailability of nitric oxide (NO) in the aorta in a stroke-prone rat model of hypertension.

### DETAILED DESCRIPTION OF THE INVENTION

At the time of filing the priority applications on which the present continuation-in-part application is based, a clear need was identified for compositions and methods for therapy and/or prevention of diseases associated with oxidative stress, such as mitochondrial oxidative stress. The invention embodiments of the priority applications address this need by providing an approach by which it is possible to use the ability of mitochondria to concentrate specific lipophilic cations to take up linked antioxidants so as to target the antioxidant to the major source of free radicals and reactive oxygen species causing the oxidative stress, for example, in therapeutic or prophylactic methods for reducing oxidative stress. As stated above, the focus of this invention is on the mitochondrial targeting of compounds, primarily for the purpose of therapy and/or prophylaxis to reduce oxidative stress. As also noted above, the present application makes explicit what was implicit in the related priority filings with respect to what the skilled person would at the time of such filings have recognized are some of the particular diseases with which oxidative stress, including mitochondrial oxidative stress, is associated. (see, e.g., Harrison's Principles of Internal Medicine, 15th Ed., Braunwald et al., eds. McGraw-Hill, New York, N.Y., 2001; Harrison's Principles of Internal Medicine, 14th Ed., Fauci et al., eds. McGraw-Hill, New York, N.Y., 1998; Harrison's Principles of Internal Medicine, 13th Ed., Kurt et al., eds. McGraw-Hill, New York, N.Y., 1994). Certain preferred embodiments as described herein relate to therapy or prophylaxis of a patient who would benefit from reduced oxidative stress wherein the patient has a liver disease characterized by elevated (e.g., increased in a statistically significant manner relative to appropriate controls) oxidative stress. Such liver diseases are described herein and include fatty liver disease (e.g., non-alcohol induced steatohepatitis, non-alcohol-induced fatty liver disease, alcohol induced steatohepatitis), hepatic viral infection (e.g., HCV), alcoholic liver disease (e.g., cirrhosis), transplant-associated liver inflammation, and cancer including cancers of the liver (e.g., hepatocellular carcinoma, HCC).

Described herein are compositions and methods for effective delivery to hepatocyte mitochondria of the presently disclosed mitochondrially targeted antioxidant compounds, including exemplary demonstrations of liver cell mitochondrial uptake of these antioxidants and their protective effects against oxidative damage. Also described herein are exemplary data showing mitochondrial uptake and cytoprotective effects of the presently disclosed mitochondrially targeted antioxidant compounds in cancer cells.

Mitochondria have a substantial membrane potential of up to 180 mV across their inner membrane (negative inside). Because of this potential, membrane permeant, lipophilic cations accumulate several-hundred fold within the mitochondrial matrix. In particular and as also noted above, where mitochondria are the major source of the free radicals and reactive oxygen species that cause oxidative stress inside most cells, it is believed that delivering antioxidants selectively to mitochondria will be more effective than using non-targeted antioxidants. Accordingly, it is towards the provision of antioxidants which may be targeted to mitochondria for the treatment and/or prevention of diseases associated with oxidative stress that certain of the presently disclosed invention embodiments are directed.

The applicants have now found that by covalently coupling lipophilic cations (preferably the lipophilic triphenylphosphonium cation) to an antioxidant the compound can be delivered to the mitochondrial matrix within intact cells. The antioxidant is then targeted to a primary production site of free radicals and reactive oxygen species within the cell, rather than being randomly dispersed.

In principle, any lipophilic cation and any antioxidant capable of being transported through the mitochondrial membrane and accumulated within the mitochondria of intact cells, can be employed in forming the compounds of the invention. It is however preferred that the lipophilic cation be the triphenylphosphonium cation herein exemplified, and that the lipophilic cation is linked to the antioxidant moiety by a carbon chain having 1 to 30 carbon atoms, preferably 1 to 20 carbon atoms.

While it is generally preferred that the carbon chain is an alkylene group (preferably C₁-C₂₀, more preferably C₁-C₁₅), carbon chains which optionally include one or more double or triple bonds are also within the scope of the invention. Also included are carbon chains which include one or more substituents (such as hydroxyl, carboxylic acid or amide groups), and/or include one or more side chains or branches, selected from unsubstituted or substituted alkyl, alkenyl or alkynyl groups.

In some particularly preferred embodiments, the linking group is an ethylene, propylene, butylene, pentylene or decylene group.

Other lipophilic cations which may covalently be coupled to antioxidants in accordance with the present invention include the tribenzyl ammonium and phosphonium cations.

Preferred antioxidant compounds of the invention, including those of general formulae I and II as defined above, can be readily prepared, for example, by the following reaction:

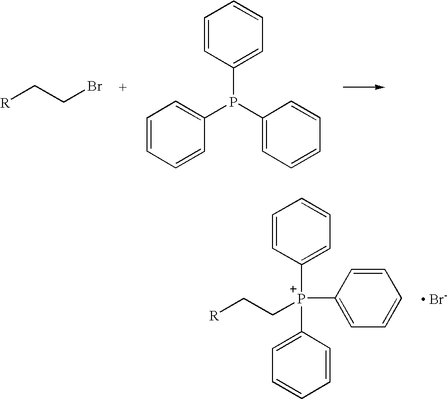

The general synthesis strategy is to heat a halogenated precursor, preferably a brominated or iodinated precursor (RBr or RI) in an appropriate solvent with 2-3 equivalents of triphenylphosphine under argon for several days. The phosphonium compound is then isolated as its bromide or iodide salt. To do this the solvent is removed, the product is then triturated repeatedly with diethyl ether until an off-white solid remains. This is then dissolved in chloroform and precipitated with diethyl ether to remove the excess triphenylphosphine. This is repeated until the solid no longer dissolves in chloroform. At this point the product is recrystallized several times from methylene chloride/diethyl ether.

It will also be appreciated that the anion of the antioxidant compound thus prepared, which will be a halogen when this synthetic procedure is used, can readily be exchanged with another pharmaceutically or pharmacologically acceptable anion, if this is desirable or necessary, using ion exchange chromatography or other techniques known in the art. Based on the disclosure herein and in the cited documents, those familiar with the art will appreciate that through such anion exchange the pharmaceutically or pharmacologically acceptable anion may in certain preferred embodiments be a pharmaceutically acceptable anion that is not a bromide ion or a nitrate anion and does not exhibit reactivity against the antioxidant moiety (e.g., as disclosed in PCT/NZ2004/000196 and PCT/NZ2004/000197), for example, an alkyl sulfonate such as methanesulfonate, p-toluenesulfonate, ethanesulfonate, benzenesulfonate, 2-naphthalenesulfonate or other alkyl sulfonate.

The same general procedure can be used to make a wide range of mitochondrially targeted compounds with different antioxidant moieties R attached to the triphenylphosphonium (or other lipophilic cationic) salt. These will include a series of vitamin E derivatives, in which the length of the bridge linking the Vitamin-E function with the triphenylphosphonium salt is varied. Other antioxidants which can be used as R include chain breaking antioxidants, such as butylated hydroxyanisole, butylated hydroxytoluene, quinols (including those of formula II as defined above) and general radical scavengers such as derivatised fullerenes. In addition, spin traps, which react with free radicals to generate stable free radicals can also be synthesized. These will include derivatives of 5,5-dimethylpyrroline-N-oxide, tert-butylnitrosobenzene, tert-nitrosobenzene, α-phenyl-tert-butylnitrone and related compounds.

In some preferred embodiments of the invention, the antioxidant compound is a quinol derivative of the formula II defined above. A particularly preferred quinol derivative of the invention is the compound mitoquinol as defined above. Another preferred compound of the invention is a compound of formula II in which (C)ₙ, is (CH₂)₅, and the quinol moiety is the same as that of mitoquinol.

Once prepared, the antioxidant compound of the invention, in any pharmaceutically appropriate form and optionally including one or more pharmaceutically-acceptable carriers, excipients and/or additives, will be administered to the patient requiring therapy and/or prophylaxis. Once administered, the compound will target the mitochondria within the cell.

The present invention thus also relates to pharmaceutical compositions containing the compounds of the invention disclosed herein. In one embodiment, the present invention relates to a composition comprising compounds of the invention in a pharmaceutically acceptable carrier, excipient or diluent and in a therapeutic amount, as disclosed herein, when administered to an animal, preferably a mammal, most preferably a human patient.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J., A. R. Gennaro, Ed., 1985).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., Eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The preferred recipients are humans, and most preferred is a human patient who would benefit from reduced oxidative stress, e.g., a patient having a disease associated with oxidative stress as provided herein.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in Kuzma et al, Regional Anesthesia 22 (6): 543-551 (1997).

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Patent No. WO 98/50016.

Current methods for ocular delivery include topical administration (eye drops), subconjunctival injections, periocular injections, intravitreal injections, surgical implants and iontophoresis (uses a small electrical current to transport ionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (oral, intravenous, inhalation, sub-cutaneous, rectal etc.), dosage forms, suitable pharmaceutical excipients and other matters relevant to the delivery of the compounds to a subject in need thereof.

Set out below are synthetic schemes which may be used to prepare some other specific mitochondrially targeted antioxidant compounds of the present invention, namely (1) a mitochondrially targeted version of buckminsterfullerene; (2) a mitochondrially targeted spin trap compound; and (3) a further synthetic route for a mitochondrially targeted spin trap compound.

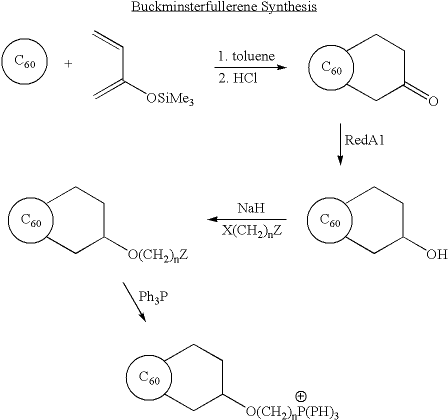

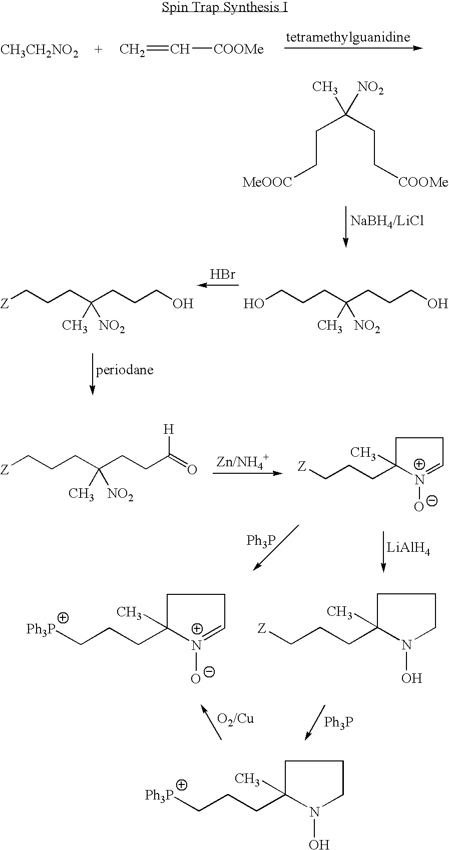

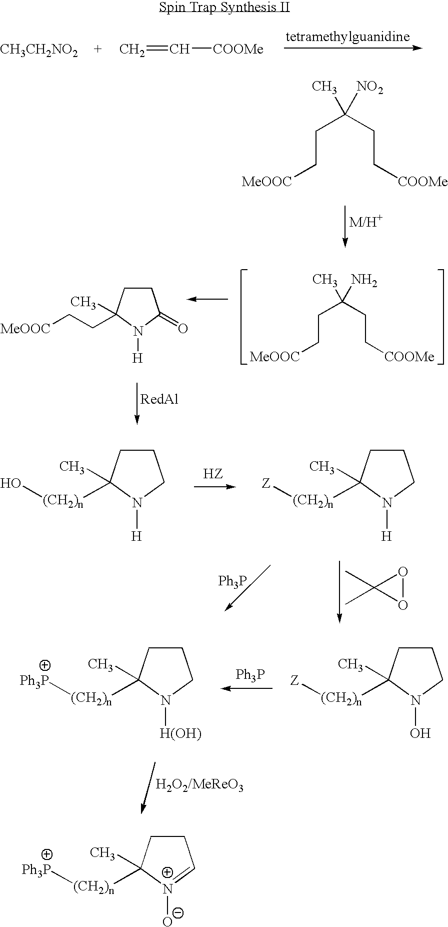

The invention will now be described in more detail with reference to the following non-limiting examples.

### EXAMPLES

### Example 1

### Experimental

### 1. Synthesis of a Mitochondrially-Targeted Vitamin-E Derivative (Compound 1)

The synthesis strategy for a mitochondrially-targeted vitamin-E derivative (compound 1) is as follows. The brominated precursor (compound 2) 2-(2-bromoethyl)-3,4-dihydro-6-hydroxy-2,5,7,8-tetramethyl-2H-1-benzopyran was synthesized by bromination of the corresponding alcohol as described by Grisar et al., (1995) (J Med Chem 38, 2880-2886). The alcohol was synthesized by reduction of the corresponding carboxylic acid as described by Cohen et al., (1979) (J. Amer Chem Soc 101, 6710-6716). The carboxylic acid derivative was synthesized as described by Cohen et al., (1982) (Syn Commun 12, 57-65) from 2,6-dihydroxy-2,5,7,8-tetramethylchroman, synthesized as described by Scott et al., (1974) (J. Amer. Oil Chem. Soc. 101, 6710-6716).

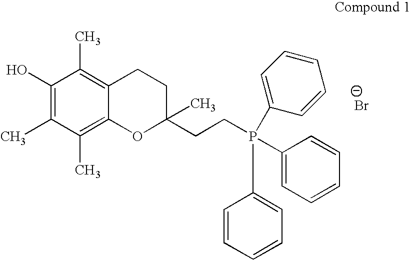

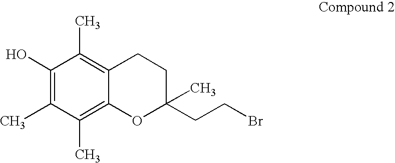

For the synthesis of compound 1,1 g of compound 2 was added to 8 ml butanone containing 2.5 molar equivalents of triphenylphosphine and heated at 100° C. in a sealed Kimax tube under argon for 7-8 days. The solvent was removed under vacuum at room temperature, the yellow oil triturated with diethyl ether until an off-white solid remained. This was then dissolved in chloroform and precipitated with diethyl ether. This was repeated until the solid was insoluble in chloroform and it was then recrystallized several times from methylene chloride/diethyl ether and dried under vacuum to give a white hygroscopic powder.

### 2. Mitochondrial Uptake of Compound 1

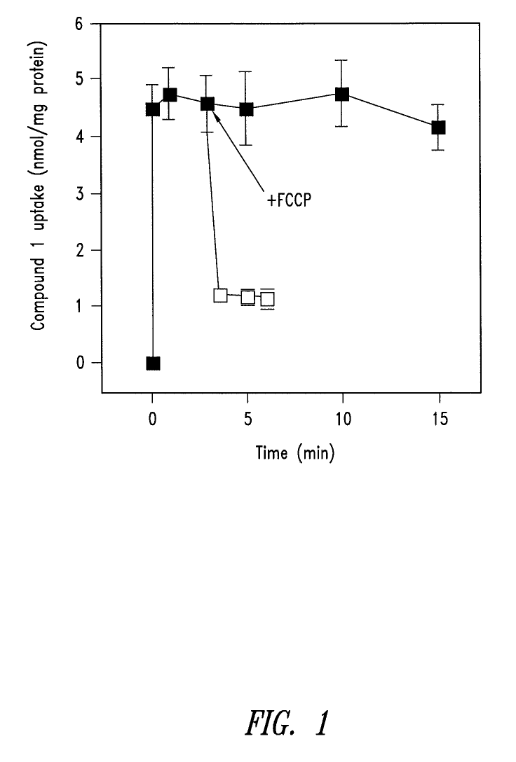

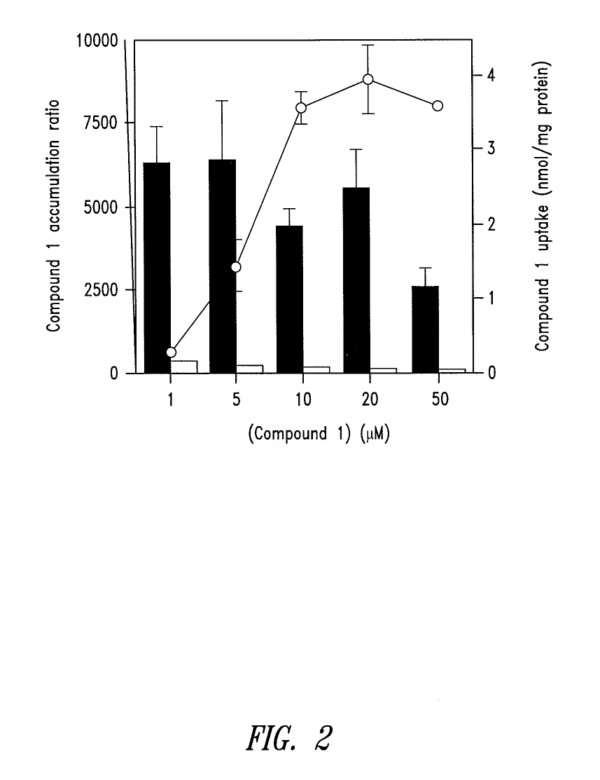

To demonstrate that this targeting is effective, the exemplary vitamin E compound 1 was tested in relation to both isolated mitochondria and isolated cells. To do this a [³H]-version of compound 1 was synthesized using [³H]-triphenylphosphine and the mitochondrial accumulation of compound 1 quantitated by scintillation counting (FIG. 1) (Burns et al., 1995, Arch Biochem Biophys 332, 60-68; Burns and Murphy, 1997, Arch Biochem Biophys 339, 33-39). To do this rat liver mitochondria were incubated under conditions known to generate a mitochondrial membrane potential of about 180 mV (Burns et al., 1995; Burns and Murphy, 1997). Under these conditions compound 1 was rapidly (<10 s) taken up into mitochondria with an accumulation ratio of about 6,000. This accumulation of compound 1 into mitochondria was blocked by addition of the uncoupler FCCP (carbonyl cyanide-p-trifluoromethoxyphenylhydrazone) which prevents mitochondria establishing a membrane potential (FIGS. 1 and 2) (Burns et al., 1995). Therefore compound 1 is rapidly and selectively accumulated into mitochondria driven by the mitochondrial membrane potential and this accumulation results in a concentration of the compound within mitochondria several thousand fold higher than in the external medium. This accumulation is rapidly (<10 s) reversed by addition of the uncoupler FCCP to dissipate the mitochondrial membrane potential after accumulation of compound 1 within the mitochondria. Therefore the mitochondrial specific accumulation is solely due to the mitochondrial membrane potential and is not due to specific binding or covalent interaction.

The mitochondrial specific accumulation of compound 1 also occurs in intact cells. This was measured as described by Burns and Murphy, 1997 and the accumulation was prevented by dissipating both the mitochondrial and plasma membrane potentials. In addition, compound 1 was not accumulated by cells containing defective mitochondria, which consequently do not have a mitochondrial membrane potential. Therefore the accumulation of compound 1 into cells is driven by the mitochondrial membrane potential.

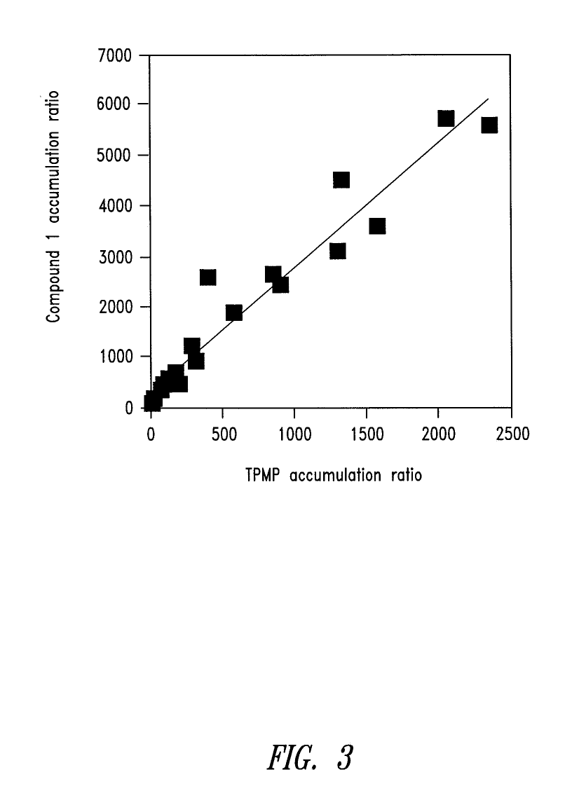

The accumulation ratio was similar across a range of concentrations of compound 1 and the amount of compound 1 taken inside the mitochondria corresponds to an intramitochondrial concentration of 4-8 mM (FIG. 2). This uptake was entirely due to the membrane potential and paralleled that of the simple triphenylphosphonium cation TPMP over a range of membrane potentials (FIG. 3). From comparison of the uptake of TPMP and compound 1 at the same membrane potential we infer that within mitochondria about 84% of compound 1 is membrane-bound (cf. About 60% for the less hydrophobic compound TPMP).

Further details of the experimental procedures and results are given below.

FIG. 1 shows the uptake of 10 μM [³H] compound 1 by energized rat liver mitochondria (continuous line and filled symbols). The dotted line and open symbols show the effect of addition of 333 nM FCCP at 3 min. Incubation with FCCP from the start of the incubation led to the same uptake as for adding FCCP at 3 min (data not shown). Liver mitochondria were prepared from female Wistar rats by homogenisation followed by differential centrifugation in medium containing 250 mM sucrose, 10 mM Tris-HCL (pH 7.4) and 1 mM EGTA and the protein concentration determined by the biuret assay using BSA as a standard. To measure [³H] compound 1 uptake mitochondria (2 mg protein/ml) were suspended at 25° C. in 0.5-1 ml 110 mM KCl, 40 mM Hepes-KOH, pH 7.2, 0.1 mM EDTA supplemented with nigericin (1 μg/ml), 10 mM succinate, rotenone 1.33 μg/ml and 60 nCi/ml [³H] compound 1 and 10 μM compound 1. After the incubation mitochondria were pelleted by centrifugation and the [³H] compound 1 in the supernatant and pellet quantitated by scintillation counting.

FIG. 2 shows the mitochondrial accumulation ratios [(compound 1/mg protein)/(compound 1 μl)] obtained following 3 min incubation of energized rat liver mitochondria with different concentrations of compound 1 (filled bars) and the effect of 333 nM FCCP on these (open bars). The dotted line and open circles show compound 1 uptake by mitochondria, corrected for FCCP-insensitive binding. To measure [³H] compound 1 accumulation ratio mitochondria (2 mg protein/ml) were suspended at 25° C. in 0.5-1 ml 110 mM KCl, 40 mM Hepes-KOH, pH 7.2, 0.1 mM EDTA supplemented with nigericin (1 μg/ml), 10 mM succinate, rotenone 1.33 μg/ml and 6-60 nCi/ml [³H] compound 1 and 1-50 μM compound 1. After the incubation mitochondria were pelleted by centrifugation and the [³H] compound 1 in the supernatant and pellet quantitated by scintillation counting.

FIG. 3 shows a comparison of compound 1 uptake with that of TPMP at a range of mitochondrial membrane potentials. Energized rat liver mitochondria were incubated for 3 min with 10 μM compound 1 and 1 μM TPMP and different membrane potentials established with 0-8 mM malonate or 333 nM FCCP. The accumulation ratios of parallel incubations with either 60 nCi/ml [³H] compound 1 or 50 nCi/ml [³H] TPMP were determined, and the accumulation ratio for compound 1 is plotted relative to that of TPMP at the same membrane potential (slope=2.472, y intercept=319, r=0.97). Mitochondria (2 mg protein/ml) were suspended at 25° C. in 0.5-1 ml 110 mM KCl, 40 mM Hepes-KOH, pH 7.2, 0.1 mM EDTA supplemented with nigericin (1 μg/ml), 10 mM succinate, rotenone 1.33 μg/ml.

### 3. Anti-Oxidant Efficacy of Compound 1

The compounds of the invention are also highly effective against oxidative stress. To demonstrate this, exemplary compound 1 was further tested using rat brain homogenates. The rat brain homogenates were incubated with or without various concentrations of the test compounds (compound 1; native Vitamin E (α-tocopherol), bromobutyl triphenylphosphonium bromide, Trolox (a water soluble form of Vitamin E) and compound 2, i.e., 2-(2-bromoethyl)-3,4-dihydro-2,5,7,8-tetramethyl-2H-1-benzopyran-6-ol, the precursor of compound 1 (“Brom Vit E”)) and the oxidative damage occurring over the incubation was quantitated using the TBARS assay (Stocks et al., 1974, Clin Sci Mol Med 47, 215-222). From this the concentration of compound required to inhibit oxidative damage by 50% was determined. In this system 210 nM compound 1 inhibited oxidative stress by 50% while the corresponding value for native Vitamin E was 36 nM. The value for bromobutyltriphenylphosphonium bromide, which contains the triphenylphosphonium moiety but not the antioxidant Vitamin E moiety was 47 μM. These data show that compound 1 is an extremely effective antioxidant, within an order of magnitude as effective as Vitamin E. Comparison with bromobutyltriphenylphosphonium bromide shows that the antioxidant capability is due to the Vitamin E function and not to the phosphonium salt. Further details of the experimental procedures and results are set out below.

The IC₅₀ values for inhibition of lipid peroxidation were determined in rat brain homogenates, and are means ±SEM or range of determinations on 2-3 brain preparations. Octan-1-ol/PBS partition coefficients are means ±SEM for three independent determinations. N.D. not determined. Partition coefficients were determined by mixing 200 μM of the compound in 2 ml water-saturated octanol-1-ol with 2 ml octanol-saturated-PBS at room temperature for 1 h, then the two layers were separated by brief centrifugation and their concentrations determined spectrophotometrically from standard curves prepared in PBS or octanol. To measure antioxidant efficacy four rat brains were homogenized in 15 ml 40 mM potassium phosphate (pH 7.4), 140 mM NaCl at 4° C., particulate matter was pelleted (1,000×g at 4° C. for 15 min) and washed once and the combined supernatants stored frozen. Aliquots were rapidly thawed and 5 mg protein suspended in 800 μl PBS containing antioxidant or ethanol carrier and incubated at 37° C. for 30 min. Thiobarbituric acid reactive species (TBARS) were quantitated at 532 nm by adding 200 μl conc. HClO₄ and 200 μl thiobarbituric acid to the incubation, heating at 100° C. for 15 min and then cooling and clarification by centrifugation (10,000×g for 2 min). The results are shown in Table 1 below.

TABLE 1
PARTITION COEFFICIENTS AND ANTIOXIDANT EFFICACY
OF COMPOUND 1 AND RELATED COMPOUNDS

| | IC₅₀ for inhibition of lipid | Octanol:PBS |
|---|---|---|
| Compound | perocidation (nM) | partition coefficient |
| Compound 1 | 210 ± 58 | 7.37 ± 1.56 |
| Bromo Vit E | 45 ± 26 | 33.1 ± 4.4 |
| α-Tocopherol | 36 ± 22 | 27.4 ± 1.0 |
| Trolox | 18500 ± 5900 | N.D. |
| BrBTP | 47000 ± 13000 | 3.83 ± 0.22 |

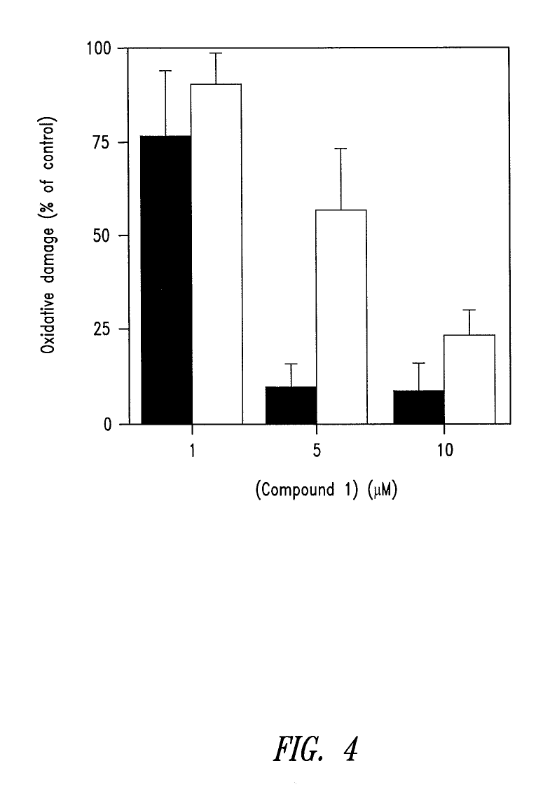

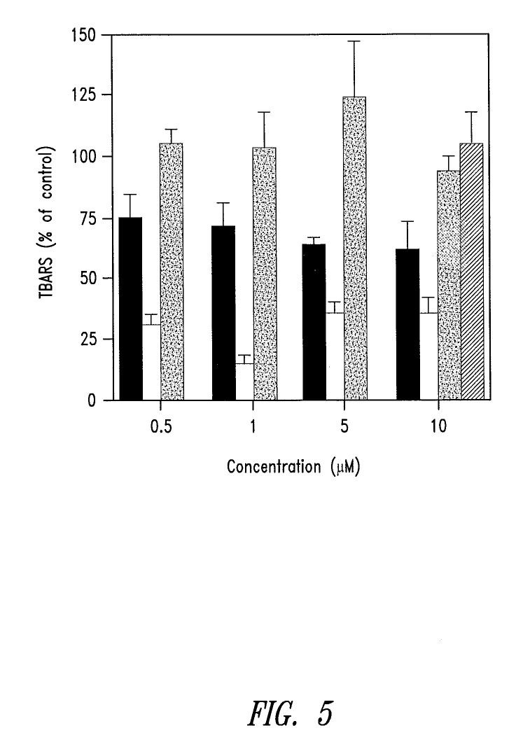

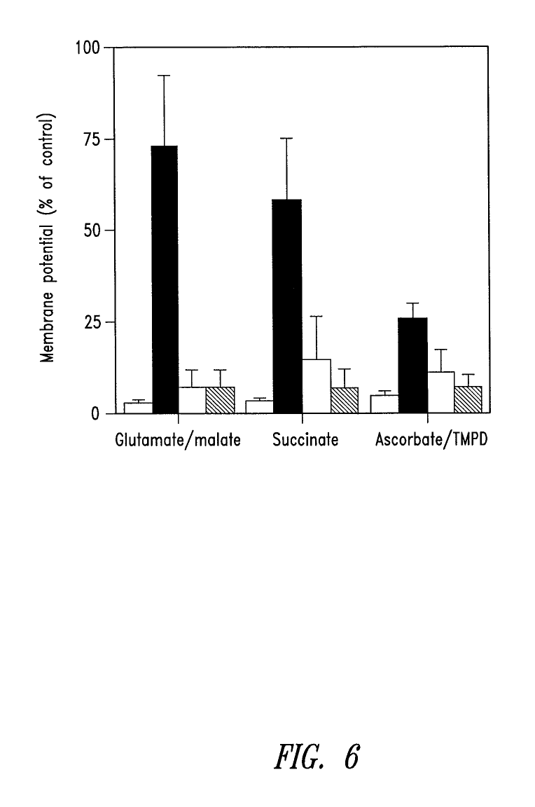

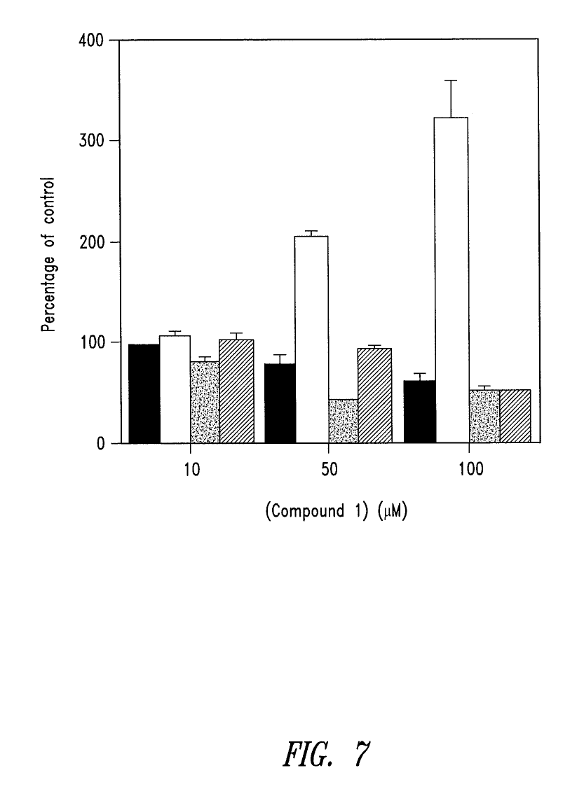

When mitochondria were exposed to oxidative stress compound 1 protected them against oxidative damage, measured by lipid peroxidation and protein carbonyl formation (FIG. 4). This antioxidant protection was prevented by incubating mitochondria with the uncoupler FCCP to prevent uptake of compound 1, and lipophilic cations alone did not protect mitochondria (FIG. 5). Most importantly, the uptake of compound 1 protected mitochondrial function, measured by the ability to generate a membrane potential, far more effectively than Vitamin E itself (FIG. 6). This shows that the accumulation of compound 1 into mitochondria selectively protects their function from oxidative damage. In addition, we showed that compound 1 is not damaging to mitochondria at the concentrations that afford protection (FIG. 7).

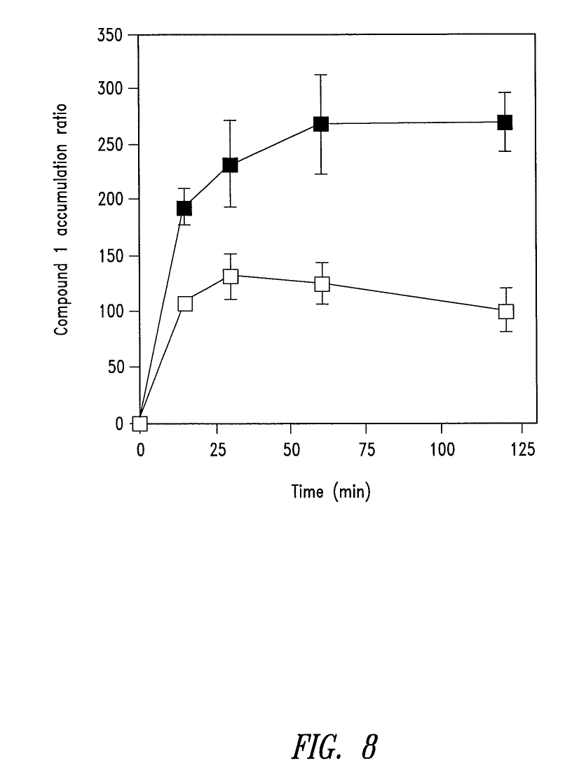

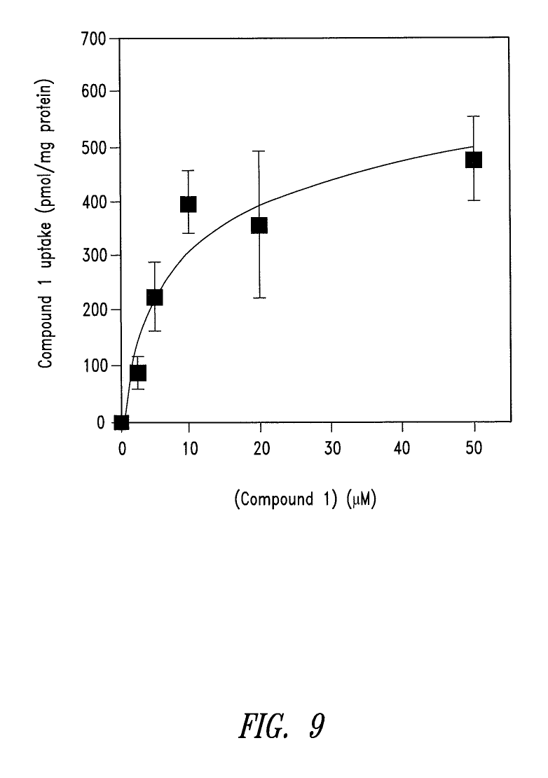

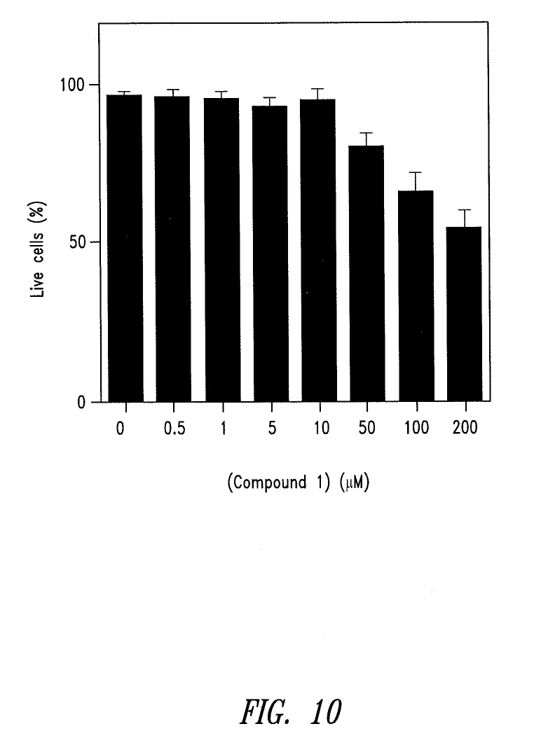

The next step was to determine whether compound 1 was accumulated by intact cells. Compound 1 was rapidly accumulated by intact 143B cells, and the amount accumulated was greater than that by ρ^{o} cells derived from 143B cells. This is important because the μ^{o} cells lack mitochondrial DNA and consequently have far lower mitochondrial membrane potential than the 143B cells, but are identical in every other way, including plasma membrane potential, cell volume and protein content (FIG. 8); this suggests that most of the compound 1 within cells is mitochondrial. A proportion of this uptake of compound 1 into cells was inhibited by blocking the plasma and mitochondrial membrane potentials (FIG. 9). This energisation-sensitive uptake corresponds to an intra mitochondrial concentration of compound 1 of about 2-4 mM, which is sufficient to protect mitochondria from oxidative damage. These concentrations of compound 1 are not toxic to cells (FIG. 10).

Further details of the experimental procedures and results are discussed below.

FIG. 4 shows the protection of mitochondria against oxidative damage by compound 1. Mitochondria were exposed to oxidative stress by incubation with iron/ascorbate and the effect of compound 1 on oxidative damage assessed by measuring TBARS (filled bars) and protein carbonyls (open bars). Rat liver mitochondria (10 mg protein) were incubated at 25° C. in a shaking water bath in 2 ml medium containing 100 mM KCl, 10 mM Tris, pH 7.7, supplemented with rotenone (1.33 μg/ml), 10 mM succinate, 500 μM ascorbate and other additions. After preincubation for 5 min, 100 μM FeSO₄ was added and 45-55 min later duplicate samples were removed and assayed for TBARS or protein carbonyls.

FIG. 5 shows a comparison of compound 1 with vitamin E and the effect of uncoupler and other lipophilic cations. Energized rat liver mitochondria were exposed to tertbutylhydroperoxide and the effect of compound 1 (filled bars), α-tocopherol (open bars), compound 1+333 nM FCCP (stippled bars) or the simple lipophilic cation bromobutyl triphenylphosphonium (cross hatched bars) on TBARS formation determined. Rat liver mitochondria (4 mg protein) were incubated in 2 ml medium containing 120 mM KCl, 10 mM Hepes-HCl pH 7.2, 1 mM EGTA at 37° C. in a shaking water bath for 5 min with various additions, then tert butyl hydroperoxide (5 mM) was added, and the mitochondria incubated for a further 45 min and then TBARS determined.

FIG. 6 shows how compound 1 protects mitochondrial function from oxidative damage. Energized rat liver mitochondria were incubated with iron/ascorbate with no additions (stippled bars), 5 μM compound 1 (filled bars), 5 μM α-tocopherol (open bars) or 5 μM TPMP (cross hatched bars), and then isolated and the membrane potential generated by respiratory substrates measured relative to control incubations in the absence of iron/ascorbate. Rat liver mitochondria were incubated at 25° C. in a shaking water bath in 2 ml medium containing 100 mM KCl, 10 mM Tris, pH 7.7, supplemented with rotenone (1.33 μg/ml), 10 mM succinate, 500 μM ascorbate and other additions. After preincubation for 5 min, 100 μM FeSO₄ was added and after 30 min the incubation was diluted with 6 ml ice-cold STE 250 mM sucrose, 10 mM Tris-HCL (pH 7.4) and 1 mM EGTA, pelleted by centrifugation (5 min at 5,000×g) and the pellet resuspended in 200 μl STE and 20 μl (=1 mg protein) suspended in 1 ml 110 mM KCl, 40 mM HEPES, 0.1 M EDTA pH 7.2 containing 1 μM TPMP and 50 nCi/ml [3H] TPMP either 10 mM glutamate and malate, 10 mM succinate and rotenone, or 5 mM ascorbate/100 μM TMPD with rotenone and myxothiazol (2 μg/ml), incubated at 25° C. for 3 min then pelleted and the membrane potential determined as above and compared with an incubation that had not been exposed to oxidative stress.

FIG. 7 shows the effect of compound 1 on the membrane potential (filled bars) and respiration rate of coupled (open bars), phosphorylating (stippled bars) and uncoupled mitochondria (cross hatched bars), as a percentage of values in the absence of compound 1. The effect of various concentrations of compound 1 on the membrane potential of isolated mitochondria was determined from the distribution of [³H] TPMP by incubating rat liver mitochondria (2 mg protein/ml) in 0.5 ml medium as above containing 1 μM TPMP and 50 nCi/ml [³H] TPMP at 25° C. for 3 min. After the incubation mitochondria were pelleted by centrifugation and the [³H] TPMP in the supernatant and pellet quantitated by scintillation counting and the membrane potential calculated assuming a volume of 0.5 μl/mg proteins and that 60% of intramitochondrial TPMP is membrane bound. To measure the effect of compound 1 on coupled, phosphorylating and uncoupled respiration rates, mitochondria (2 mg protein/ml) were suspended in 120 mM KCl 10 mM Hepes-HCl pH 7.2, 1 mM EGTA, 10 mM K Pi in a 3 ml Clark oxygen electrode then respiratory substrate, ADP (200 μM) and FCCP (333 nM) were added sequentially to the electrode and respiration rates measured.

FIG. 8 shows the uptake of compound 1 by cells. Here 10⁶ 143B cells (closed symbols) or ρ^{o} cells (open symbols) were incubated with 1 μM [3H] compound 1 and the compound 1 accumulation ratio determined. Human osteosarcoma 143B cells and a derived ρ^{o} cell line lacking mitochondrial DNA were cultured in DMEM/10% FCS (foetal calf serum) supplemented with uridine and pyruvate under an atmosphere of 5% CO₂/95% air at 37° C., grown to confluence and harvested for experiments by treatment with trypsin. To measure [³H] compound 1 accumulation cells (10⁶) were incubated in 1 ml HEPES-buffered DMEM. At the end of the incubation, cells were pelleted by centrifugation, the cell pellet and the supernatant prepared for scintillation counting and the accumulation ratio [compound 1/mg protein)/(compound 1/μl)] calculated.

FIG. 9 shows the amount of compound 1 taken up by 10⁶ 143B cells over 1 h incubation, corrected for inhibitor-insensitive binding. Human osteosarcoma 143B cells were incubated in 1 ml HEPES-buffered DMEM with 1-50 μM compound 1 supplemented with 6-60 nCi/ml [³H] compound 1. To determine the energistration-dependent uptake, parallel incubations with 12.5 μM oligomycin, 20 μM FCCP, 10 μM myxathiazol, 100 nM valinomycin and 1 mM ouabain were carried out. At the end of the incubation, cells were pelleted by centrifugation and prepared for scintillation counting and the energisation-sensitive uptake determined.

FIG. 10 shows the effect of compound 1 on cell viability. Here, confluent 143B cells in 24 well tissue culture dishes were incubated with various concentrations of compound 1 for 24 h and cell viability measured by lactate dehydrogenase release.

### Example 2

### Synthesis of [10-(6′-ubiquinonyl)decyltriphenylphosphonium bromide] (herein referred to as “mitoquinol”)

### Synthesis of Precursors

To synthesize 11-bromoundecanoic peroxide 11-bromoundecanoic acid (4.00 g, 15.1 mmol) and SOCl₂ (1.6 mL, 21.5 mmol) were heated, with stirring, at 90° C. for 15 min. Excess SOCl₂ was removed by distillation under reduced pressure (15 mm Hg, 90° C.) and the residue (IR, 1799 cm⁻¹) was dissolved in diethyl ether (20 mL) and the solution cooled to 0° C. Hydrogen peroxide (30%, 1.8 mL) was added, followed by dropwise addition of pyridine (1.4 mL) over 45 min. Diethyl ether (10 mL) was added and the mixture was stirred for 1 h at room temperature then diluted with diethyl ether (150 mL) and washed with H₂O (2×70 mL), 1.2 M HCl (2×70 mL), H₂O (70 mL), 0.5 M NaHCO₃ (2×70 mL) and H₂O (70 mL). The organic phase was dried over MgSO₄ and the solvent removed at room temperature under reduced pressure, giving a white solid (3.51 g). IR (nujol mull) 1810, 1782.

6-(10-bromodecyl)ubiquinone was synthesized by mixing crude material above (3.51 g, 12.5 mmol max), (ubiquinoneₒ, 1.31 g, 7.19 mmol, Aldrich) and acetic acid (60 mL) and stirring the mixture for 20 h at 100° C. The mixture was diluted with diethyl ether (600 mL) and washed with H₂O (2×400 mL), 1 M HCl (2×450 mL), 0.50 M NaHCO₃ (2×450 mL) and H₂O (2×400 mL). The organic phase was dried over MgSO₄. The solvent was removed under reduced pressure, giving a reddish solid (4.31 g). Column chromatography of the crude solid on silica gel (packed in CH₂Cl₂) and elution with CH₂Cl₂ gave the product as a red oil (809 mg, 28%) and unreacted ubiquinone as a red solid (300 mg, 1.6 mmol, 13%). TLC: R_{f} (CH₂Cl₂, diethyl ether 20:1) 0.46; IR (neat) 2928, 2854, 1650, 1611, 1456, 1288; λₘₐₓ (ethanol): 278 nm; ¹H NMR (299.9 MHz) 3.99 (s, 6H, 2-OCH₃), 3.41 (t, J=6.8 Hz, 3H, —CH₂—Br), 2.45 (t, J=7.7 Hz, 2H, ubiquinone-CH₂—), 2.02, (s, 3H, —CH₃). 1.89 (quin, J=7.4 Hz, 3H, —CH₂—CH₂—Br), 1.42-1.28 (m, 20H, —(CH₂)₇—); ¹³C NMR (125.7 MHz) 184.7 (carbonyl), 184.2 (carbonyl), 144.3 (2C, ring), 143.1 (ring), 138.7 (ring), 61.2 (2×-OCH₃), 34.0 (—CH₂—), 32.8 (—CH₂—), 29.8 (—CH₂—), 29.4 (2×-CH₂—), 29.3 (—CH₂—), 28.7 (2×-CH₂—), 28.2 (—CH₂—), 26.4 (—CH₂—), 11.9 (—CH₃), Anal. Calcd. For C₁₉H₂₉O₂Br:C, 56.86; H, 7.28. Found: C, 56.49, H, 7.34; LREI mass spectrum: calcd. For C₁₉H₂₉O₂Br 400/402. Found 400/402.

To form the quinol, 6-(10-bromodecyl)-ubiquinol, NaBH₄ (295 mg, 7.80 mmol) was added to a solution of the quinone (649 mg, 1.62 mmol) in methanol (6 mL) and stirred under argon for 10 min. Excess NaBH₄ was quenched with 5% HCl (2 mL) and the mixture diluted with diethyl ether (40 mL). The organic phase was washed with 1.2 M HCl (40 mL) and saturated NaCl (2×40 mL), and dried over MgSO₄. The solvent was removed under reduced pressure, giving a yellow oily solid (541 mg, 83%). ¹H NMR (299.9 MHz) 5.31 (s, 1H, —OH), 5.26 (s, 1H, —OH), 3.89 (s, 6H, 2×-OCH₃), 3.41 (t, J=6.8 Hz, 2H, —CH₂—Br), 2.59 (t, J=7.7 Hz, 2H ubquinol-CH₂—), 2.15 (s, 3H, CH₃) 1.85 (quin, J=7.4 Hz, 2H, —CH₂—CH₂—Br), 1.44-1.21 (m, 19H, —CH₂)₇—).

### Synthesis of 10-(6′-ubiguinonyl)decyltriphenylphosphonium bromide (‘mitoquinol’)

To synthesize 10-(6′-ubiquinolyl)decyltriphenylphosphonium bromide. To a 15 mL Kimax tube were added 6-(10-bromodecyl)ubiquinol (541 mg, 1.34 mmol), PPH₃ (387 mg, 1.48 mmol), ethanol (95%, 2.5 mL) and a stirring bar. The tube was purged with argon, sealed and the mixture stirred in the dark for 88 h at 85° C. The solvent was removed under reduced pressure, giving an oily orange residue. The residue was dissolved in CH₂Cl₂ (2 mL) followed by addition of pentane (20 mL). The resultant suspension was refluxed for 5 min at 50° C. and the supernatant decanted. The residue was dissolved in CH₂Cl₂ (2 mL) followed by addition of diethyl either (20 mL). The resultant suspension was refluxed for 5 min at 40° C. and the supernatant decanted. The CH₂Cl₂/diethyl ether reflux was repeated twice more. Residual solvent was removed under reduced pressure, giving crude product as a cream solid (507 mg). ¹H NMR (299.9 MHz) 7.9-7.6 (m, 20H, —P⁺Ph₃), 3.89 (s, 6H, 2×-OCH₃), 3.91-3.77 (m, 2H, —CH₂—P+Ph₃), 2.57 (t, J=7.8 Hz, 2H ubquinol-CH₂—), 2.14 (s, 3H, CH₃), 1.6-1.2 (m, 23H, —(CH₂)₈—). ³¹P NMR (121.4 MHz) 25.1.

The crude product (200 mg) was oxidized to 10-(6′-ubiquinonyl)decyltriphenylphosphonium bromide (the oxidized form) by stirring in CDCl₃ under an oxygen atmosphere for 13 days. The oxidation was monitored by ¹H NMR and was complete after 13 days. The solvent was removed under reduced pressure and the resultant residue dissolved in CH₂Cl₂ (5 mL). Excess diethyl ether (15 mL) was added and the resultant suspension stirred for 5 min. The supernatant was decanted and the CH₂Cl₂/diethyl ether precipitation repeated twice more. Residual solvent was removed under reduced pressure, giving crude product as a brown sticky solid (173 mg).

The quinone was reduced to the quinol by taking a mixture of crude quinone and quinol (73 mg, ca. 3:1 by ¹H NMR) in methanol (1 mL) was added NaBH₄ (21 mg, 0.55 mmol). The mixture was stirred slowly under an argon atmosphere for 10 min. Excess NaBH₄ was quenched with 5% HBr (0.2 mL) and the mixture extracted with CH₂Cl₂. The organic extract was washed with H₂O (3×5 mL). Solvent was removed under reduced pressure, giving a mixture of quinone and quinol (ca 1:5 by ¹H NMR) as a pale yellow solid (55 mg). For routine preparation of the quinol form the ethanolic solution, dissolve in 5 vols of water, (=1 ml) add a pinch of NaBH₄ leave on ice in the dark for 5 min, then extract 3×0.5 ml dichloromethane, wash with water/HCl etc blow off in nitrogen, dissolve in same vol of etoh and take spectrum and store at −80 under argon. Yield about 70-80%. Oxidizes rapidly in air so should be prepared fresh. Vortex with 1 ml 2M NaCl. Collect the upper organic phase and evaporate to dryness under a stream of N₂ and dissolve in 1 ml ethanol acidified to pH 2.

### Synthesis of [³H]-10-(6′-ubiguinonyl)decyltriphenylphosphonium bromide

To a Kimax tube was added 6-(10-bromodecyl)ubiquinol (6.3 mg; 15.6 μmol) triphenylphosphine (4.09 mg; 15.6 μmol) and 100 μl ethanol containing [³H] triphenylphosphine (74 μCi custom synthesis by Moravek Biochemicals, Brea, Calif., USA, Spec Ac 1 Ci/mmol) and 150 μl ethanol added. The mixture was stirred in the dark under argon for 55 h at 80° C. Then it was cooled and precipitated by addition of 5 ml diethyl ether. The orange solid was dissolved in a few drops of dichloromethane and then precipitated with diethyl ether and the solid was washed (×4) with ˜2 ml diethyl ether. Then it was dissolved in ethanol to give a stock solution of 404 μM which was stored at −20° C. The UV absorption spectrum and TLC were identical to those of the unlabeled 10-(6′-ubiquinonyl)decyltriphenylphosphonium bromide and the specific activity of the stock solution was 2.6 mCi/mmol.

### Extinction Coefficients

Stock solutions of the quinone in ethanol were stored at −80° C. in the dark and their concentrations confirmed by 31P nmr. The compound was converted to the fully oxidized form by incubation in basic 95% ethanol over an hour on ice or by incubation with beef heart mitochondrial membrane at room temperature, either procedure leading to the same extinction coefficient of 10,400 M⁻¹ cm⁻¹ at the local maximum of 275 nm, with shoulders at 263 and 268 nm corresponding to the absorption maxima of the triphenylphosphonium moiety (Smith et al., Eur. J. Biochem., 263, 709-716, 1999; Burns et al., Archives of Biochemistry and Biophysics, 322, 60-68, 1995) and a broad shoulder at 290 nm due to the quinol (Crane et al., Meth. Enzymol., 18C, 137-165, 1971). Reduction by addition of NaBH₄ gave the spectrum of the quinol which had the expected peak at 290 nm with an extinction coefficient of 1800 M⁻¹ cm⁻¹ and the extinction coefficient for at 268 nm was 3,000 M⁻¹ cm⁻¹ the same as that for the phosphonium moiety alone (Burns, 1995 above). The extinction coefficient of 10,400M⁻¹ cm⁻¹ at 275 nm was lower than that for other quinones which have values of 14,600 M⁻¹ cm⁻¹ in ethanol (Crane, 1971 above) and 12,250 M⁻¹ cm⁻¹ in aqueous buffer (Cabrini et al., Arch. Biochem Biophys, 208, 11-19, 1981). While the absorbance of the quinone was about 10% lower in buffer than in ethanol, the discrepancy was not due to an interaction between the phosphonium and the quinone as the absorbance of the precursor quinone before linking to the phosphonium and that of the simple phosphonium methyltriphenylphosphonium were additive when 50 μM of each were mixed together in either ethanol or aqueous buffer. The Δε_{ox-red} was 7,000 M⁻¹ cm⁻¹.

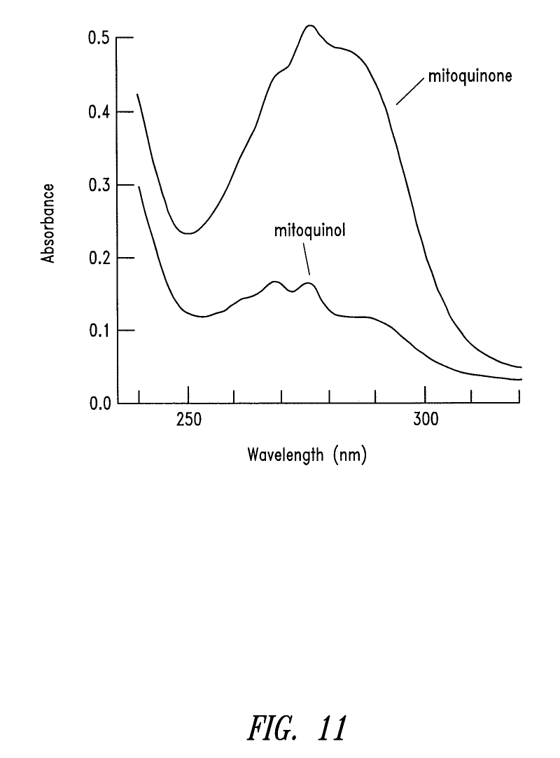

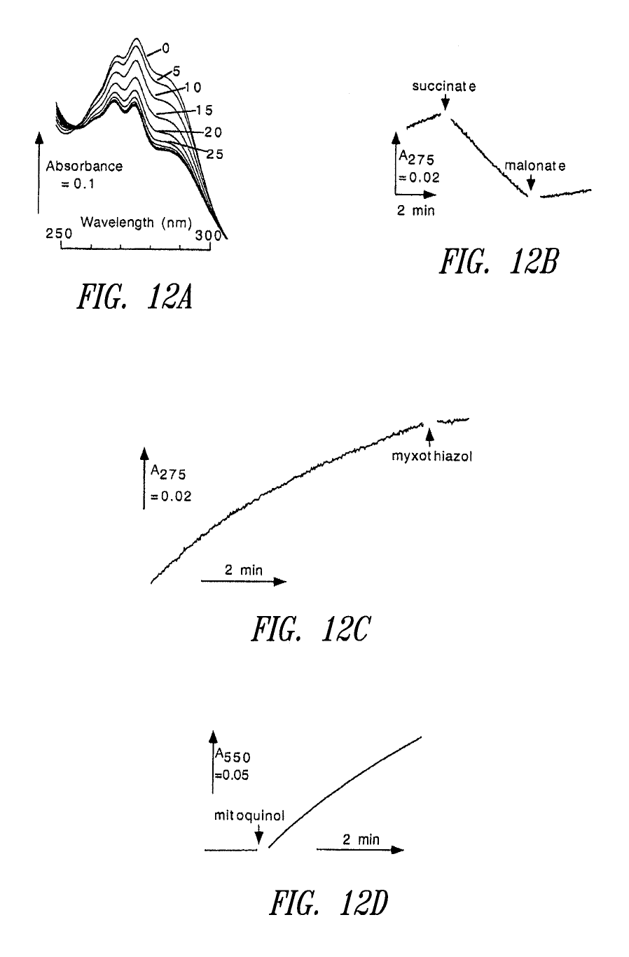

The spectrum of fully oxidized mitoquinone (50 μM) in 50 mM sodium phosphate, pH 7.2 is shown in FIG. 11. Addition of NaBH₄ gave the fully reduced compound, mitoquinol. The UV absorption spectrum of the reduced (quinol) and oxidized (quinone) mitoquinone/ol are shown in FIG. 11. To determine whether the mitochondrial respiratory chain could also oxidise or reduce the compound mitoquinone was incubated with beef heart mitochondrial membranes (FIG. 12). In panel A the spectrum of fully oxidized mitoquinone in the presence of antimycin inhibited membranes is shown (t=0; FIG. 12A). Addition of succinate led to the gradual reduction of the mitoquinol as measured by repeating the measurement every five minutes and showing that the peak at 275 nm gradually disappeared, the presence of antimycin prevented the oxidation of the quinol by mitochondrial complex III. Succinate did not lead to the complete reduction of mitoquinone to mitoquinol, as can be seen by comparing the complete reduction brought about by borohydride (FIG. 11), instead it reduced about 23% of the added ubiquinone (FIG. 12A). This is presumably due to equilibration of the quinol/quinone couple with the succinate/fumarate couple (Em Q=40 mV at pH 7, Em Suc=30 mV), hence this proportion corresponds to an Eh of about +8 mV.

The reduction of mitoquinone can be followed continuously at A₂₇₅ nm (FIG. 12B). On addition to rotenone inhibited mitochondrial membranes the small amount of mitoquinol remaining was oxidized leading to a slight increase in A₂₇₅, but on addition of the Complex II substrate succinate mitoquinone was rapidly reduced and this reduction was blocked by malonate, an inhibitor of Complex II (FIG. 12B). The rate of reduction of mitoquinone was 51±9.9 nmol/min/mg protein, which compares with the rate of reduction of cytochrome c by succinate in the presence of KCN of 359 nmol/min/mg. Allowing for the 2 electrons required for mitoquinone reduction compared with 1 for cytochrome c the rate of electron flux into the mitoquinone pool is of similar order to the electron flux through the respiratory chain.

To determine whether mitoquinol was oxidized by Complex III of the respiratory chain, mitoquinol was added to beef heart membranes which had been inhibited with rotenone and malonate (FIG. 12C). The mitoquinol was oxidized rapidly by membranes at an initial rate of about 89±9 nmol mitoquinol/min/mg protein (mean of 2+/−range) and this oxidation was blocked by myxathiazol an inhibitor of complex III (FIG. 12C). To confirm that these electrons were being passed on to cytochrome c, mitoquinol was then added to membrane supplemented with ferricytochrome c and the rate of reduction of cytochrome c monitored (FIG. 12D). Addition of mitoquinol led to reduction of cytochrome c at an initial rate of about 93+/−13 nmol/min/mg (mean +/−range). This rate was largely blocked by myxathiazol, although a small amount of cytochrome c reduction (about 30-40%) was not blocked by myxathiazol.

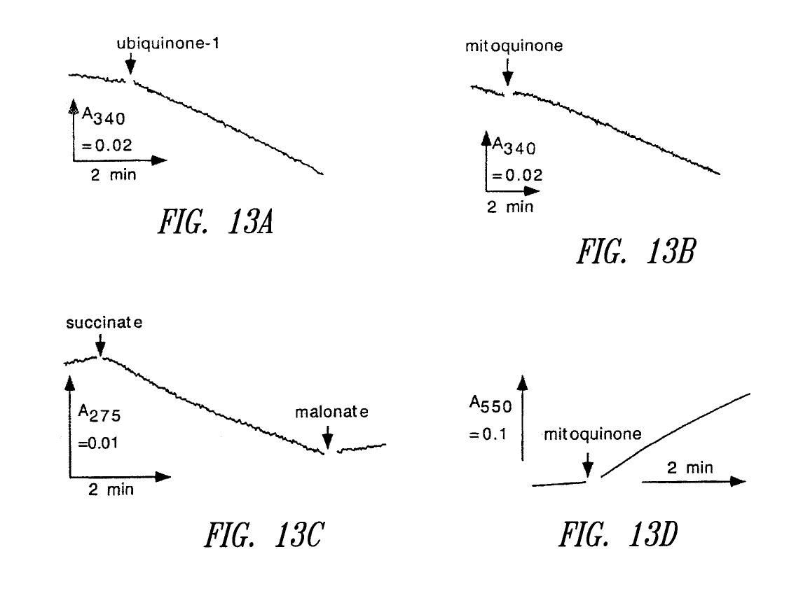

Mitoquinone/ol may be picking up and donating electrons directly from the active sites of the respiratory complexes, or it could be equilibrating with the endogenous mitochondrial ubiquinone pool. To address this question the endogenous ubiquinone pool was removed from beef heart mitochondria by pentane extraction. In the absence of endogenous ubiquinone as an electron acceptor the pentane extracted beef heart mitochondria could not oxidise added NADH, but addition of ubiquinone-1, a ubiquinone analog that can pick up electrons from the active site of complex 1, the oxidation of NADH is partially restored (FIG. 13A). Similarly, addition of mitoquinone also restored NADH oxidation indicating that mitoquinone can pick up electrons from the complex I active site (FIG. 13B). Succinate could also donate electrons to mitoquinone in pentane extracted beef heart mitochondrial in a malonate sensitive manner, suggesting that mitoquinone could also pick up electrons from the active site of Complex II (FIG. 13C). Finally, the effect of the quinone on the flux of electrons to cytochrome c was determined and it was shown that there was no NADH-ferricytochrome c activity until mitoquinone was added (FIG. 13D), and this was partially inhibited by myxathizol (60-70%).

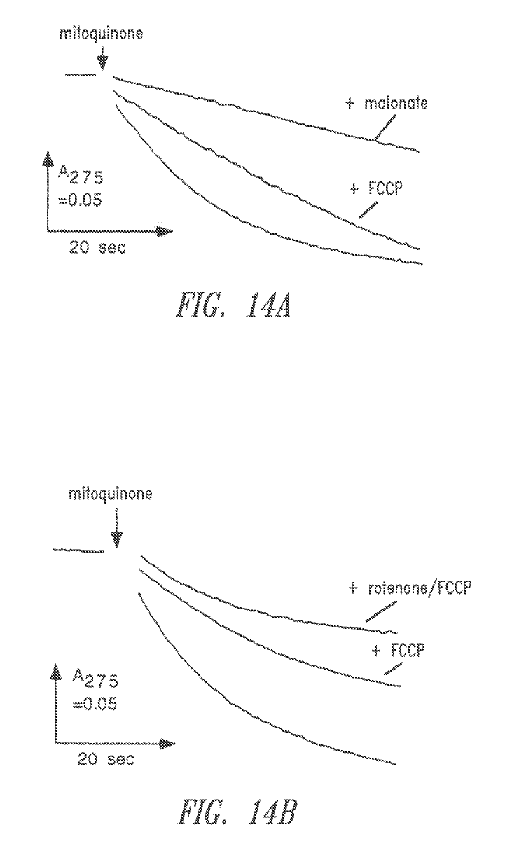

The next step was to see if mitoquinone also accepted electrons within intact mitochondria (FIG. 14). When mitoquinone was added to intact energized mitochondria it was rapidly reduced (FIG. 14A). In the presence of the uncoupler FCCP to dissipate the membrane potential the rate was decreased about 2-3 fold, presumably due to the prevention of the uptake of the compound in to the mitochondria (FIG. 14A). The complex II inhibitor malonate also decreased the rate of reduction of mitoquinone (FIG. 14A). Use of the NADH-linked substrates glutamate/malate also led to the rapid reduction of mitoquinone by intact mitochondria which again was decreased by addition of the uncoupler FCCP (FIG. 14B). The Complex I inhibitor rotenone also decreased the rate of reduction of mitoquinone (FIG. 14B).

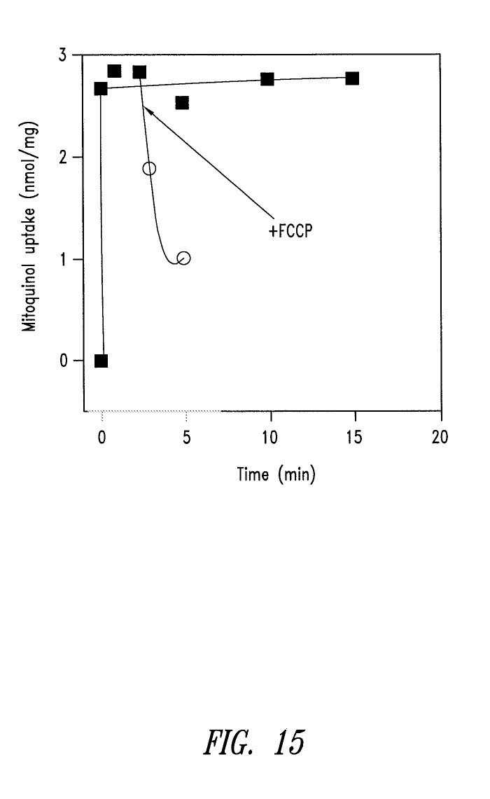

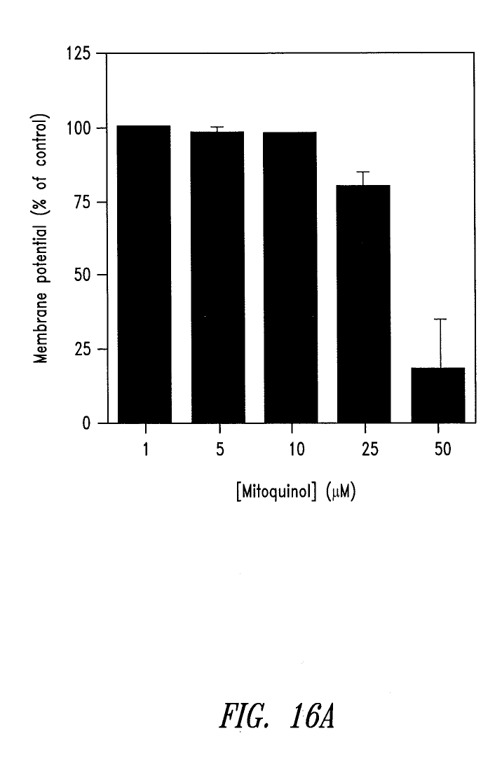

The next step was to see if mitoquinol was accumulated by energized mitochondria. To do this a tritiated version of the compound was made, incubated with energized mitochondria and the amount taken up into the mitochondria determined. It can be seen that the compound is accumulated rapidly and that this accumulation is reversed by addition of the uncoupler FCCP (FIG. 15).

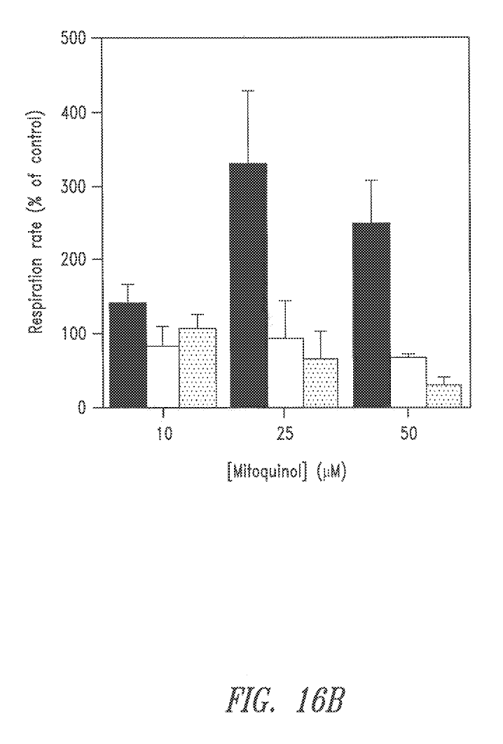

The next assays were to determine the toxicity of these compounds to mitochondria and cells. To determine the toxicity to isolated mitochondria the effect on membrane potential and respiration rate were measured (FIG. 16). It can be seen from FIG. 16 that 10 μM mitoquinol had little effect on mitochondrial function and at 25 μM and above there was some uncoupling and inhibition of respiration.

### Example 3

### Mitochondrial Antioxidant 10-(6′-ubiquinonyl) decyltriphenylphosphonium IN Chronic Hepatitis C Virus (HCV) Infection

A double-blind, randomized, parallel design trial was conducted to compare the effects of two different doses (40 mg/day or 80 mg/day) of MitoQuinone [10-(6′-ubiquinonyl)decyltriphenylphosphonium methanesulfonate] and of placebo in patients with documented history of chronic HCV infection. Participants were randomized to receive either 40 mg, 80 mg or matching placebo for 28 days. Randomization was in a 1:1:1 ratio in permutated blocks of 6. The inclusion criteria for subjects were as follows: (i) between 18 and 65 years of age; (ii) chronic hepatitis C infection (any genotype); (iii) non-responders, intolerant for treatment with current standard-of-care (PEGylated interferon plus ribavirin); (iiv) plasma ALT level of 2-10 times the upper limit of normal (ULN) at study entry; (v) Metavir Stage 0, 1, or 2 (no evidence of cirrhosis); (vi) alcohol intake of less than 5 standard drinks/week; (vii) no recent use of anti-oxidants (e.g., Co-enzyme-Q). The subject group baseline demographics were as follows:

Placebo group: 7 males, 3 females, age 46 years, BMI 28.0, 7 Caucasian, 3 Asian/Pacific, liver fibrosis stage 0 or 1 (7 subjects) and stage 2 (3 subjects), HCV viral load 3.6×10⁶, 10 years duration, interferon experience 3 intolerant, 4 unsuitable, 6 failed; baseline ALT 155.2, baseline AST 96.8.

MitoQuinone (40 mg/day; 0.5 mg/kg) group: 6 males, 5 females, age 48 years, BMI 25.6, 10 Caucasian, 1 Asian/Pacific, liver fibrosis stage 0 or 1 (4 subjects) and stage 2 (7 subjects), HCV viral load 2.4×10⁶, 9 years duration, interferon experience 1 intolerant, 4 unsuitable, 6 failed; baseline ALT 153.2, baseline AST 103.6.

MitoQuinone (80 mg/day; 1 mg/kg) group: 6 males, 3 females, age 49 years, BMI 28.1, 7 Caucasian, 2 Asian/Pacific, liver fibrosis stage 0 or 1 (2 subjects) and stage 2 (5 subjects), HCV viral load 2.4×10⁶, 11 years duration, interferon experience 1 intolerant, 2 unsuitable, 5 failed; baseline ALT 130.9, baseline AST 87.4.

Efficacy Endpoints were (i) percentage change in plasma ALT concentration at Day 28 compared with baseline; (ii) absolute change in HCV RNA viral load at Day 28 compared with baseline; (iii) plasma levels of MitoQuinone for population pharmacokinetics. The analyses were on intention-to-treat population (at least one dose of study medication and at least one post-dosing liver function test). A last observation carried forward (LOCF) was utilized for premature terminations. ANOVA was used to compare treatment groups.

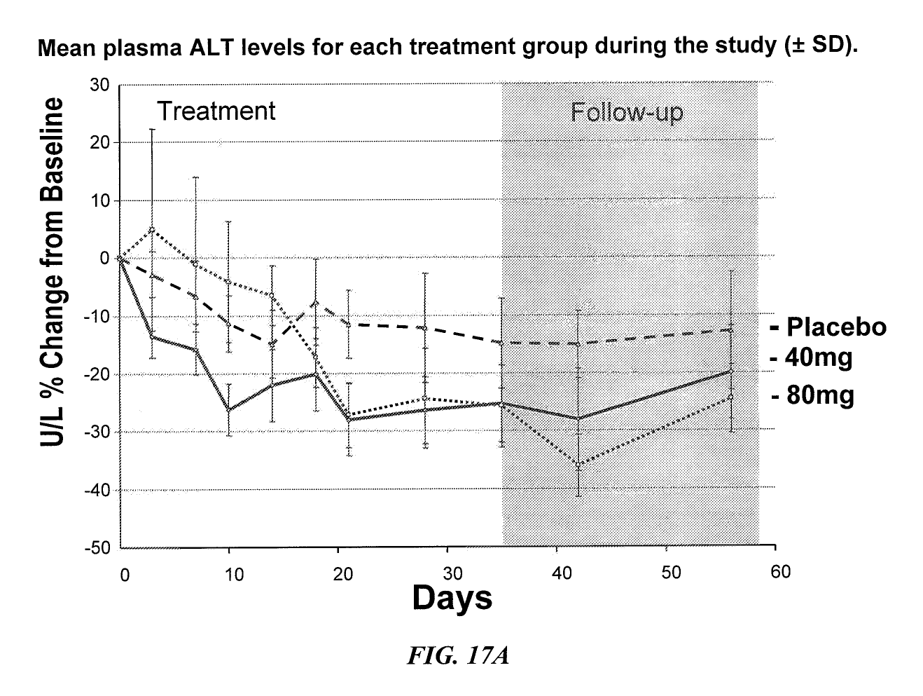

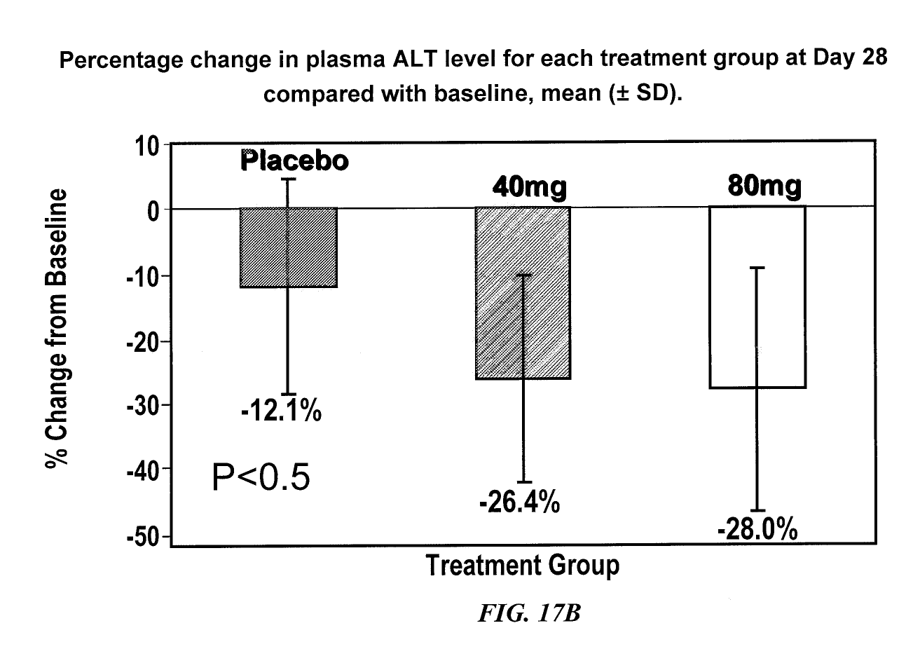

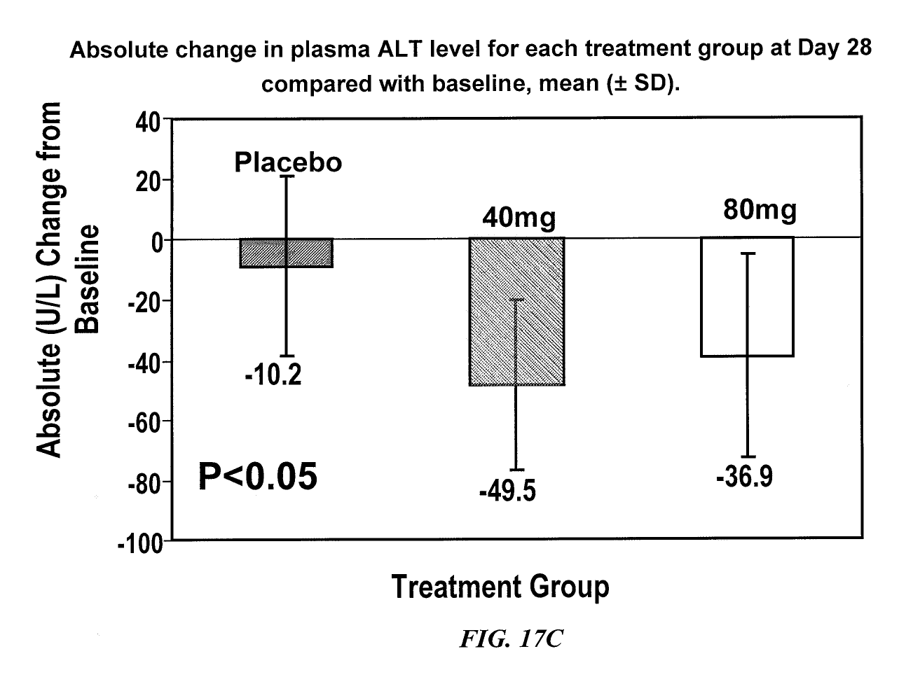

Using the EPP analysis (n=29 participants), the primary efficacy results demonstrated a significant decrease in both percentage and absolute change in plasma ALT at Day 28 compared with baseline for both the 40 mg and 80 mg treatment groups (P<0.05) (FIG. 17). FIG. 17 shows plasma alanine aminotransferase (ALT) levels in chronically HCV-infected subjects receiving indicated dose of 10-(6′-ubiquinonyl) decyltriphenylphosphonium methanesulfonate or placebo during (“treatment”) and after (“follow-up”) a treatment regimen. FIG. 17A shows mean plasma ALT levels (U/L % change from baseline) over time during the daily treatment period and, after daily treatments were stopped, through a follow-up period. FIG. 17B shows the percentage change in plasma ALT level for each treatment group at day 28, compared to baseline levels. FIG. 17C shows the absolute change in plasma ALT level for each treatment group at day 28, compared to baseline.

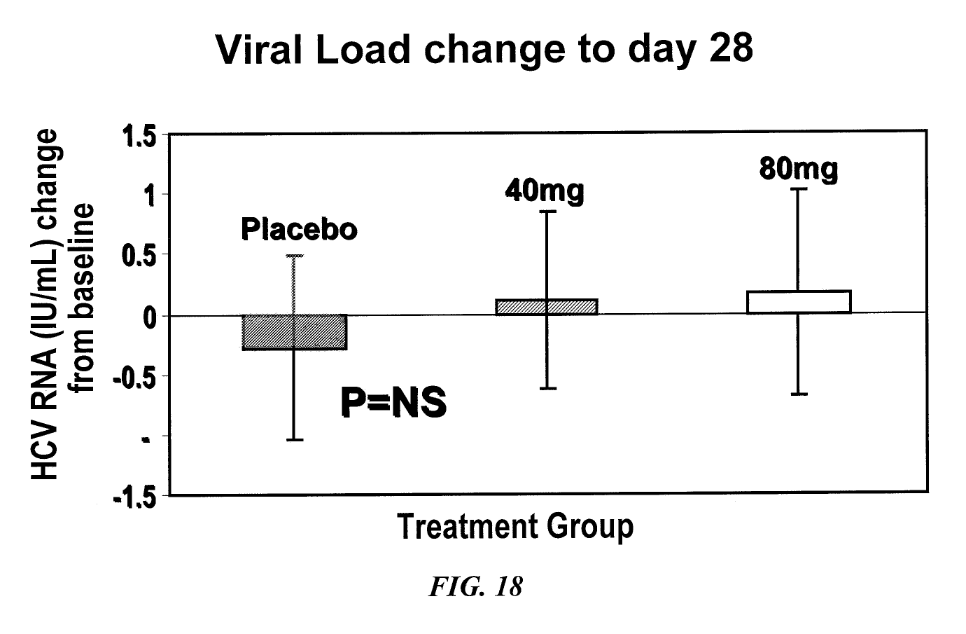

Serum HCV RNA levels did not change significantly during the study in any treatment group (FIG. 18). FIG. 18 shows HCV viral load as determined from HCV RNA detection (by a standard PCR-based assay using HCV RNA-specific oligonucleotide probes) in chronically HCV-infected subjects receiving the indicated dose of 10-(6′-ubiquinonyl) decyltriphenylphosphonium methanesulfonate or placebo during a treatment regimen.

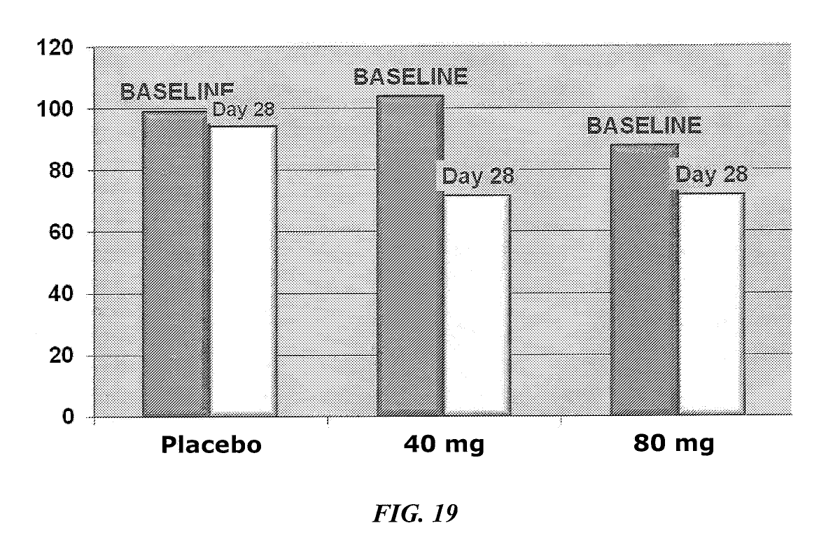

FIG. 19 shows the mean change, compared to baseline levels, in plasma aspartate aminotransferase (AST) levels in chronically HCV-infected subjects receiving indicated dose of 10-(6′-ubiquinonyl) decyltriphenylphosphonium methanesulfonate or placebo at day 28 during a treatment regimen.

### Example 4

### Mitochondrial Antioxidant 10-(6′-ubiquinonyl) decyltriphenylphosphonium IN Cardiovascular Disease Models

This example describes the effects of MitoQuinone [10-(6′-ubiquinonyl) decyltriphenylphosphonium methanesulfonate] using the stroke-prone spontaneously hypertensive (SHRSP) rat model of hypertension, which has been previously described (McIntyre et al., 1997 Hypertension 30:1517; Alexander et al., 1999 Cardiovasc. Res. 43:798; Fennell et al., 2002 Gene Ther. 9:110).

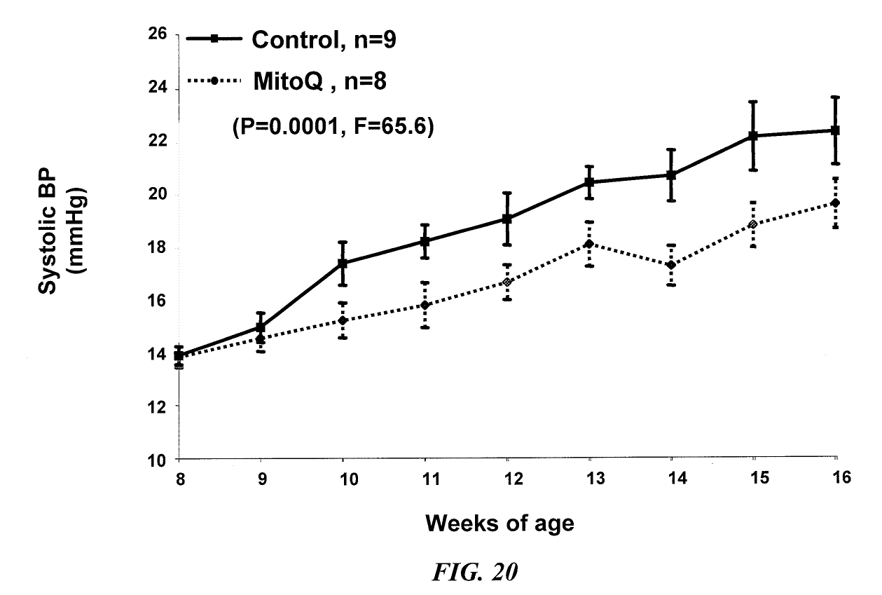

First, using animals and assay methodologies as previously described (e.g., McIntyre et al., 1997; Alexander et al., 1999; Fennell et al., 2002) groups (8-9 animals/group) of 8-week old male SHRSP rats were treated either with MitoQuinone (0.5 or 1 mg/kg) or vehicle control over a period of 8 weeks, and systolic blood pressure measurements were recorded at weekly intervals. As shown in FIG. 20, animals treated with 10-(6′-ubiquinonyl) decyltriphenyl-phosphonium exhibited significant reductions of systolic blood pressure in this spontaneously hypertensive stroke-prone rat model.

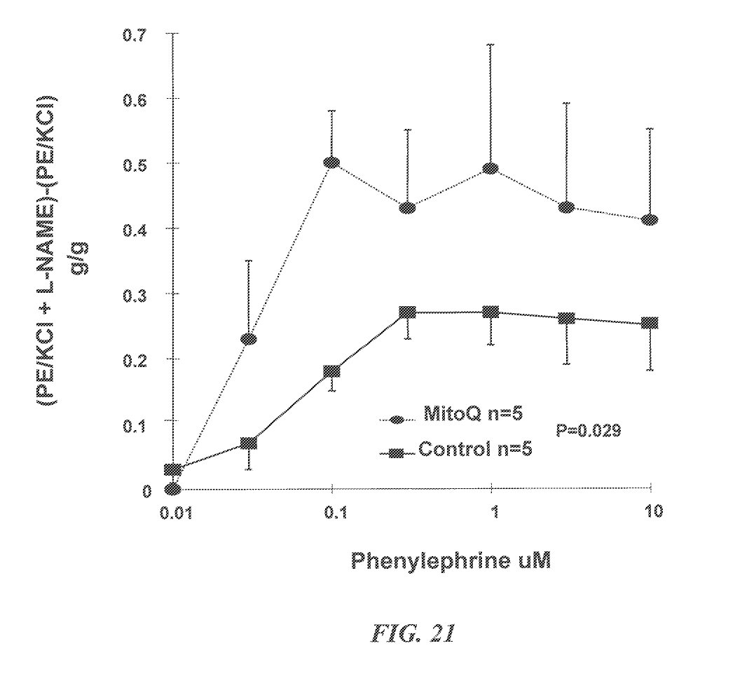

Next, control and MitoQuinone-treated SHRSP rats were used as sources of aortic rings in an assay for bioavailable nitric oxide (NO) essentially according to the procedures described by McIntyre et al. (1997) and Alexander et al. (1999). Briefly, basal NO bioavailability was measured by the change in isometric tension of aortic rings in response to phenylephrine (PE) after addition of the NOS inhibitor N^{ω}-nitro-L-arginine methyl ester (L-NAME, 100 μmol/L) to the organ bath. FIG. 21 shows the effects of 10-(6′-ubiquinonyl) decyltriphenyl-phosphonium on the bioavailability of nitric oxide (NO) in the aortas of spontaneously hypertensive SHRSP rats.

### INDUSTRIAL APPLICATION

The compounds of the invention have application in selective antioxidant therapies for human patients to prevent mitochondrial damage. This can be to prevent the elevated mitochondrial oxidative stress associated with particular diseases, such as a fatty liver disease (e.g., non-alcohol induced steatohepatitis, non-alcohol-induced fatty liver disease, alcohol-induced steatohepatitis), a hepatic viral infection (e.g., HCV), alcoholic liver disease (e.g., cirrhosis), transplantation-associated liver inflammation, liver cancer (e.g., hepatocellular carcinoma) or other cancer (e.g., carcinoma, osteosarcoma, etc.), cardiometabolic syndrome, a cardiovascular disease characterized by elevated oxidative stress (e.g., hypertension, atherosclerosis, heart failure), macular or retinal degeneration, anthracycline-induced cardiotoxicity, sepsis (e.g., septic shock or endotoxic shock), obstructive pulmonary disease, cystic fibrosis, emphysema, pulmonary fibrosis, adult respiratory distress syndrome, pulmonary hypertension, asbestosis, chronic obstructive pulmonary disease, type 1 diabetes, type 2 diabetes, impaired glucose tolerance, diabetic neuropathy, amyotrophic lateral sclerosis, Parkinson's disease, Huntington's disease, Alzheimer's disease, Freidreich's ataxia, traumatic brain injury, or diseases associated with mitochondrial DNA mutations (see, e.g., Beal, Howell and Bodis-Wollner (Eds.), Mitochondria & Free Radicals in Neurodegenerative Diseases, 1997, Wiley-Liss, NY). They could also be used in conjunction with cell transplant therapies for neurodegenerative diseases, to increase the survival rate of implanted cells.

In addition, these compounds could be used as prophylactics to protect organs during transplantation, or ameliorate the ischemia-reperfusion injury that occurs during surgery. The compounds of the invention could also be used to reduce cell damage following stroke and heart attack or be given prophylactically to premature babies, which are susceptible to brain ischemia. The methods of the invention have a major advantage over current antioxidant therapies they will enable antioxidants to accumulate selectively in mitochondria, the part of the cell under greatest oxidative stress. This will greatly increase the efficacy of antioxidant therapies. Related lipophilic cations are being trailed as potential anticancer drugs and are known to be relatively non-toxic to whole animals, therefore these mitochondrially-targeted antioxidants are unlikely to have harmful side effects. For example, the presently disclosed mitochondrially targeted antioxidant compounds are contemplated for use in therapeutic and/or prophylactic methods for treating, preventing or impairing the progression of neoplastic and/or malignant diseases, including methods that comprise administering to a patient a mitochondrially targeted antioxidant and an anticancer drug (e.g., an anthracyline drug such as doxorubicin, daunomycin, hydroxyldaunorubicin, etc., or other anticancer drugs such as bleomycin, taxotere, vincristine, vinblastine, cisplatin, etoposide, 5-FU, etc.) whereby, according to non-limiting theory, the herein disclosed mitochondrially targeted antioxidant compound potentiates the anticancer activity of the anticancer drug.

Those persons skilled in the art will appreciate that the above description is provided by way of example only, and that different lipophilic cation/antioxidant combinations can be employed without departing from the scope and spirit of the invention embodiments as disclosed herein.

What is claimed is:

1. A method of therapy or prophylaxis of a patient who would benefit from reduced oxidative stress, comprising:
administering to the patient a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the patient has a disease that is selected from the group consisting of:
(i) a human degenerative disease associated with aging,
(ii) non-specific cell, tissue or organ damage that accumulates with aging,
(iii) inflammation,
(iv) ischemia-reperfusion tissue injury accompanying at least one of stroke, heart attack, organ transplantation and surgery,
(v) diabetes,
(vi) neurodegenerative disease, and
(vii) cancer.

2. A method of therapy or prophylaxis of a patient who would benefit from reduced oxidative stress, comprising:
administering to the patient a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the patient has a disease that is selected from the group consisting of:
(i) a liver disease characterized by elevated oxidative stress,
(ii) a cardiometabolic syndrome condition characterized by elevated oxidative stress,
(iii) a cardiovascular disease characterized by elevated oxidative stress,
(iv) macular or retinal degeneration,
(v) anthracycline-induced cardiotoxicity,
(vi) sepsis, and
(vii) a lung disease characterized by elevated oxidative stress.

3. The method of either claim 1 or claim 2 wherein oxidative stress comprises mitochondrial oxidative stress.

4. The method of claim 1 wherein:
(i) the human degenerative disease associated with aging is selected from the group consisting of Parkinson's disease and Alzheimer's disease,
(ii) the inflammation is caused by sepsis or septic shock,
(iii) the diabetes comprises at least one condition that is selected from type 1 diabetes, type 2 diabetes, impaired glucose tolerance and a diabetic complication,
(iv) the neurodegenerative disease is selected from amyotrophic lateral sclerosis, Parkinson's disease, Huntington's disease, Alzheimer's disease, Freidreich's ataxia and traumatic brain injury, and
(v) the cancer comprises hepatocellular carcinoma.

5. The method of claim 4 wherein sepsis or septic shock comprises endotoxic shock.

6. The method of claim 4 wherein the diabetic complication comprises diabetic neuropathy.

7. The method of claim 2 wherein the liver disease characterized by elevated oxidative stress is selected from a fatty liver disease, a hepatic viral infection, alcoholic liver disease, transplantation-associated liver inflammation and liver cancer.

8. The method of claim 7 wherein the fatty liver disease is selected from non-alcohol induced steatohepatitis, non-alcohol-induced fatty liver disease, and alcohol-induced steatohepatitis.

9. The method of claim 7 wherein the hepatic viral infection comprises a hepatitis C virus (HCV) infection.

10. The method of claim 7 wherein the liver cancer comprises hepatocellular carcinoma.

11. The method of claim 2 wherein the cardiovascular disease characterized by elevated oxidative stress comprises one or more of cardiovascular hypertension, atherosclerosis and heart failure.

12. The method of claim 2 wherein the lung disease characterized by elevated oxidative stress is selected from obstructive pulmonary disease, cystic fibrosis, emphysema, pulmonary fibrosis, adult respiratory distress syndrome, pulmonary hypertension and asbestosis.

13. The method of claim 12 wherein the obstructive pulmonary disease is chronic obstructive pulmonary disease.

14. A method of treating or preventing a disease associated with oxidative stress, comprising:
administering a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the disease associated with oxidative stress is selected from the group consisting of:
(i) a human degenerative disease associated with aging,
(ii) non-specific cell, tissue or organ damage that accumulates with aging,
(iii) inflammation,
(iv) ischemia-reperfusion tissue injury accompanying at least one of stroke, heart attack, organ transplantation and surgery,
(v) diabetes,
(vi) neurodegenerative disease, and
(vii) cancer.

15. A method of treating or preventing a disease associated with oxidative stress, comprising:
administering a mitochondrially-targeted antioxidant compound comprising a lipophilic cation covalently coupled to an antioxidant moiety, wherein the antioxidant moiety is capable of being transported through a mitochondrial membrane and accumulated within mitochondria of intact cells, wherein the compound is not thiobutyltriphenylphosphonium bromide, and wherein the disease associated with oxidative stress is selected from the group consisting of:
(i) a liver disease characterized by elevated oxidative stress,
(ii) a cardiometabolic syndrome condition characterized by elevated oxidative stress,
(iii) a cardiovascular disease characterized by elevated oxidative stress,
(iv) macular or retinal degeneration,
(v) anthracycline-induced cardiotoxicity,
(vi) sepsis, and
(vii) a lung disease characterized by elevated oxidative stress.

16. The method of either claim 14 or claim 15 wherein oxidative stress comprises mitochondrial oxidative stress.

17. The method of claim 14 wherein:
(i) the human degenerative disease associated with aging is selected from the group consisting of Parkinson's disease and Alzheimer's disease,
(ii) the inflammation is caused by sepsis or septic shock,
(iii) the diabetes comprises at least one condition that is selected from type 1 diabetes, type 2 diabetes, impaired glucose tolerance and a diabetic complication,
(iv) the neurodegenerative disease is selected from amyotrophic lateral sclerosis, Parkinson's disease, Huntington's disease, Alzheimer's disease, Freidreich's ataxia and traumatic brain injury, and
(v) the cancer comprises hepatocellular carcinoma.

18. The method of claim 17 wherein sepsis or septic shock comprises endotoxic shock.

19. The method of claim 17 wherein the diabetic complication comprises diabetic neuropathy.

20. The method of claim 15 wherein the liver disease characterized by elevated oxidative stress is selected from a fatty liver disease, a hepatic viral infection, alcoholic liver disease, transplantation-associated liver inflammation and liver cancer.

21. The method of claim 20 wherein the fatty liver disease is selected from non-alcohol induced steatohepatitis, non-alcohol-induced fatty liver disease, and alcohol-induced steatohepatitis.

22. The method of claim 20 wherein the hepatic viral infection comprises a hepatitis C virus (HCV) infection.

23. The method of claim 20 wherein the liver cancer comprises hepatocellular carcinoma.

24. The method of claim 15 wherein the cardiovascular disease characterized by elevated oxidative stress comprises one or more of cardiovascular hypertension, atherosclerosis and heart failure.

25. The method of claim 15 wherein the lung disease characterized by elevated oxidative stress is selected from obstructive pulmonary disease, cystic fibrosis, emphysema, pulmonary fibrosis, adult respiratory distress syndrome, pulmonary hypertension and asbestosis.

26. The method of claim 25 wherein the obstructive pulmonary disease is chronic obstructive pulmonary disease.
